(19) United States
(12) Patent Application Publication
(10) Pub. No.: US 2023/0087756 A1
(43) Pub. Date: Mar. 23, 2023

(54) SYSTEMS AND METHODS FOR GENERATING HOMOGENOUS MIXTURES OF BREWED BEVERAGES AND ACTIVE INGREDIENTS

(71) Applicant: RECEPTOR HOLDINGS, INC., Seattle, WA (US)

(72) Inventors: Paul N. Camera, Sammamish, WA (US); Gregory Wesner, Bainbridge Island, WA (US)

(73) Assignee: RECEPTOR HOLDINGS, INC., Seattle, WA (US)

(21) Appl. No.: 17/790,503

(22) PCT Filed: Dec. 31, 2020
(86) PCT No.: PCT/US2020/067645

§ 371 Date: Jun. 30, 2022

Related U.S. Application Data
(60) Provisional application No. 62/956,572, filed on Jan. 2, 2020.

(51) Int. Cl. A61K 31/352; A61K 31/05; A61K 31/60; A61K 9/00; A61K 31/07; A61K 36/9066; A61K 31/12; A47J 31/06; A47J 31/46; B65D 85/804; A23F 5/26

(52) U.S. Cl. CPC A61K 31/352; A61K 31/05; A61K 31/60; A61K 9/0095; A61K 31/07; A61K 36/9066; A61K 31/12; A47J 31/06; A47J 31/468; B65D 85/8046; A23F 5/267; A47J 31/461

(57) ABSTRACT

Various techniques for generating substantially homogenous mixtures of nonpolar materials and aqueous solutions are disclosed herein. An example method includes generating a substantially homogenous mixture of an aqueous solution and a nonpolar material by flowing the aqueous solution through a porous substrate infused with the nonpolar material.

### CROSS-REFERENCE TO RELATED APPLICATION

This application claims the benefit of U.S. Provisional Application No. 62/956,572, titled “SYSTEMS AND METHODS FOR GENERATING HOMOGENOUS MIXTURES OF BREWED BEVERAGES AND ACTIVE INGREDIENTS” and filed on Jan. 2, 2020, which is incorporated by reference herein in its entirety.

### FIELD OF THE DISCLOSURE

The current disclosure provides systems, methods, and uses of aqueous solutions, such as brewed beverages, containing nonpolar active ingredients.

### BACKGROUND OF THE DISCLOSURE

Coffee is a brewed beverage that is prepared from roasted berries of various species of the genus Coffea, such as Coffea arabica and Coffea robusta. In various examples, berries can be dried and roasted. The roasted berries (also referred to as “beans”) can be ground and brewed with water to generate coffee. Coffee can have various tastes due to the type of plant from which the berries are selected, the technique by which the berries are roasted, the method by which water is introduced to the coffee, or the like. Due to its caffeine content and desirable taste, coffee and other coffee-based beverages are popular throughout the world.

Coffee can be mixed with various additives to generate new tastes and effects. Coffee can be served with milk (e.g., café latte, mélange, or the like), cream, other dairy products, or dairy substitutes. Some individuals believe that the taste of coffee can be improved with sugar. In various cases, individuals enjoy coffee combined with various syrups (e.g., chocolate syrup, vanilla syrup, or the like) that can add additional flavors to their coffee beverages. In addition, coffee can be served with mood-altering ingredients, such as alcoholic ingredients (e.g., whiskey, brandy, or the like). Because of coffee's ubiquity, individuals have sought to enjoy coffee with various other flavors, supplements, and active ingredients.

Cannabinoids are active ingredients that have a number of desirable effects when consumed. Cannabinoids are a diverse class of compounds that interact with and activate cannabinoid receptors. There are three classes of cannabinoids: 1) endocannabinoids, which are naturally produced in the body by humans and other animals, 2) phytocannabinoids, which are produced by plants, and 3) synthetic cannabinoids, which are chemically produced cannabinoids. Synthetic cannabinoids can be identical to cannabinoids that are found in nature or can be compounds that do not exist in nature.

Endocannabinoids are part of the endocannabinoid system, which refers to endogenous cannabinoids and cannabinoids receptors. Cannabinoid receptors are expressed in various cell types, including brain cells and immune cells. An example of an endocannabinoid is anandamide, which is a fatty acid neurotransmitter that interacts with cannabinoid receptors and is involved in regulating the sensations of hunger, motivation, and pleasure.

Phytocannabinoids are produced by various types of plants, but the most well-known cannabinoid-producing plant is Cannabis. Cannabis sativa is an example of a plant in the Cannabis genus. Other plants of the Cannabis genus include Cannabis indica and Cannabis ruderalis. Hybrids between Cannabis sativa and Cannabis indica are common. Cannabis plants produce over 100 cannabinoids, many of which have therapeutic potential. The most well-known cannabinoids produced by Cannabis include (−)-trans-delta-9-tetrahydrocannabinol (THC, THC), cannabidiol (CBD) and cannabinol. Other cannabinoids found in plants of the Cannabis genus include cannabigerol, cannabidivarin, tetrahydrocannabivarin, and cannabichromene.

Plants other than Cannabis are known to produce cannabinoids. Examples of such plants include Echinacea purpurea, Echinacea angustifolia, Acmella oleracea, Helichrysum umbraculigerum, and Radula marginata. Cannabinoids isolated from plants in the Echinacea genus include lipophilicalkamides (alkylamides). Over 25 different alkylamides have been identified. They include the cis/trans isomers dodeca-2E,4E,8Z,10E/Z-tetraenoic-acid isobutylamide.

THC, the primary psychoactive compound of Cannabis, is the most extensively studied cannabinoid and has many well-established health benefits. THC is prescribed under the pharmaceutical drug name dronabinol, and is U.S. Food and Drug Administration (FDA)-approved for use as an appetite stimulant for HIV and AIDS-related weight loss and for chemotherapy-induced nausea and vomiting. Many other medical uses of THC are being investigated, and research indicates that THC may have anti-tumor activity (Guzman M, Nat Rev Cancer. 2003. 3:745-55), anti-inflammatory effects (Gaiffal E, et al. Allergy. 2013. 68(8): 994-1000), and analgesic effects (Pharm. J. 259, 104, 1997 and in Pharm. Sci. 3, 546, 1997).

THC exhibits complex effects on the central nervous system (CNS), including central sympathomimetic activity. THC demonstrates effects on mood, cognition, memory, appetite and perception. These effects appear to be dose related. After most forms of oral administration, THC has an onset of action of 0.5 to 1 hour and a peak effect at 2-4 hours. The duration of action for psychoactive effects is 4-6 hours, but the appetite stimulant effect may continue for 24 hours or longer after administration. THC is almost completely absorbed (90-95%) after single oral doses.

Nabilone, a synthetic cannabinoid not found in nature, is another cannabinoid that has numerous medical uses. Nabilone, which is structurally very similar to THC, has been reported to be an anti-emetic and anxiolytic, and is also useful for treating pain of various etiologies such as multiple sclerosis (MS), peripheral neuropathy and spinal injuries (Lancet, 1995, 345, 579, Pharm. J. 259, 104, 1997; Baker & Pryce, Expert Opin Investig Drugs. 2003 April; 12(4):561-7).

Other formulations of THC, such as THC-containing tinctures and extracts, also have medicinal and recreational uses (Peschel, Sci. Pharm. 2016, 84(3):567-584). Tinctures and extracts can include an alcoholic extract of Cannabis, or components of Cannabis, such as THC. Nabiximols, for example, is a Cannabis extract that contains a one-to-one ratio of cannabidiol (CBD):THC and is commercially available as Sativex® (GW Pharmaceuticals plc, Wilshire, United Kingdom). Nabiximols is used to treat spasticity (muscle spasms and stiffness) in MS patients.

Cannabinoids are, in general, nonpolar. Despite the promising applications of cannabinoid-containing formulations, it is difficult to mix cannabinoids with aqueous solutions. Similarly, it is difficult to mix other nonpolar molecules with aqueous solutions. For instance, when a nonpolar material (e.g., a fluid containing a cannabinoid) is added to an aqueous solution (e.g., coffee), the nonpolar material will spontaneously float to the top of the aqueous solution, thereby generating a separated layer. The separated layer is generally undesirable, at least in part because the taste and the texture of the separated layer can be objectionable to drinkers.

### SUMMARY OF THE DISCLOSURE

The current disclosure describes various techniques for producing homogenous mixtures of nonpolar materials and aqueous solutions. For example, various techniques disclosed herein can be used to produce mixtures of cannabinoids and coffee-based beverages, such as espresso-based beverages. In particular implementations, a porous substrate (e.g., a paper substrate, a ceramic substrate, or the like) can be infused with a nonpolar material (e.g., a material containing a nonpolar molecule). A pressure can be applied to an aqueous solution, such that the aqueous solution flows through the infused porous substrate. As the aqueous solution flows through the porous substrate, the nonpolar material can be mixed with the aqueous solution. Accordingly, a homogenous mixture (e.g., an emulsion) of the aqueous solution and the nonpolar material can be generated.

In some implementations, various techniques disclosed herein can be used to produce homogenous mixtures of brewed beverages and nonpolar materials. For example, a porous substrate infused with a nonpolar material can be placed in a brewing receptacle, such as a drip coffee machine basket, a portafilter, a French press, a pour over basket, a beverage pod, or the like. As used herein, the terms “portafilter,” “porta-filter,” and their equivalents, can refer to a component of an espresso machine configured to hold ground coffee beans, receive heated water, and to expel brewed espresso. For example, the portafilter includes a basket configured to hold the ground coffee beans and that includes holes configured to pass the brewed espresso without passing the ground coffee beans. In some cases, the porous substrate can be in the presence of ground coffee beans and/or tea leaves. When pressurized water flows through the ground coffee beans and/or tea leaves and the porous substrate, a brewed beverage (e.g., coffee or tea) infused with the nonpolar molecule can be generated. The pressurized water may generate a relatively homogenous mixture of the nonpolar molecule in the brewed beverage. Accordingly, a brewed beverage (e.g., a coffee-based beverage) containing a nonpolar material can be generated without the nonpolar material separating into a distinct layer (e.g., at the top or bottom of the beverage) due to the immiscibility of the nonpolar material with the brewed beverage.

Various nonpolar materials can be mixed with aqueous solutions, according to various implementations described herein. In particular examples, a cannabinoid (e.g., CBD, THC, or the like) can be infused in a porous substrate and homogenously mixed with an aqueous solution by flowing the aqueous solution through the porous substrate. According to some implementations, other nonpolar molecules, such as nonpolar flavor- and/or aroma-conferring molecules, nonpolar supplemental materials (e.g., vitamins, minerals, or other additives), and the like, can be infused into a porous substrate and mixed with aqueous solutions using various techniques described herein.

The current disclosure also provides various systems for producing homogenous mixtures of nonpolar molecules and aqueous solutions, as well as methods for manufacturing homogenous mixtures, for administering homogenous mixtures to individuals (e.g., patients, consumers, or the like), for treating symptoms of diseases or disorders using homogenous mixtures, and for treating diseases or disorders using homogenous mixtures.

### BRIEF DESCRIPTION OF THE FIGURES

FIG. 1A illustrates an example system for producing homogenous cannabinoid-containing coffee beverages.

FIG. 1B is a cross-sectional diagram illustrating an example system for producing a homogenous mixture of coffee and a nonpolar molecule using a beverage pod.

FIG. 2 illustrates an example environment for generating a cannabinoid-infused filter for producing homogenous cannabinoid-containing expresso beverages.

FIG. 3 provides exemplary structures of cannabinoids that can be synthetically derived (e.g., THC, nabilone, CBD, 7-OH-CBD, CBDV, 7-OHCBDV).

FIG. 4 provides various cannabis-derived molecules such as terpenes and flavonoids.

FIG. 5 illustrates results of an experimental example wherein homogenous CBD-containing espresso beverages were prepared with different amounts of CBD.

### DETAILED DESCRIPTION

The current disclosure provides brewed beverages containing one or more nonpolar molecules (e.g., cannabinoids). In various implementations of the disclosure, brewed beverages include a mixture of at least one nonpolar molecule and coffee or tea. In some examples, brewed beverages include CBD and/or espresso.

According to various implementations, an aqueous fluid (e.g., water, espresso, or the like) can be pressed through a porous substrate infused with a nonpolar molecule. In some cases, the porous substrate can include paper, ceramic, pulp, or the like. A pressure by which the aqueous fluid is pressed through the infused porous substrate can mix the aqueous fluid with at least one nonpolar molecule infused in and/or on the porous substrate. Accordingly, a substantially homogenous mixture (e.g., an emulsion) of the aqueous fluid and the nonpolar molecule(s) can be generated, regardless of the difference in polarity between the aqueous fluid and the nonpolar molecule(s).

In various examples, a porous substrate can be infused with at least one nonpolar molecule (e.g., a cannabinoid). In some implementations, a porous substrate can be exposed to a fluid including at least one nonpolar molecule (e.g., an oil including a cannabinoid). In some cases, the fluid includes the nonpolar molecule(s) and an additional solvent (e.g., an oil or an alcohol, such as ethanol) in which the nonpolar molecule(s) are suspended. According to various examples, the porous substrate may include one or more spaces (e.g., pores) by which the fluid including the nonpolar molecule(s) can spontaneously propagate through the porous substrate via the capillary effect. For instance, the porous substrate may include paper, pulp, ceramic, or the like. In some cases, the porous substrate may be dried before the porous substrate is used to infuse an aqueous fluid (e.g., water, coffee, tea, or the like) with the nonpolar molecule(s).

As used herein, the term “composition,” and its equivalents, can refer to a fluid including at least one nonpolar molecule. Examples of compositions include a substantially homogenous mixture of an aqueous fluid and a nonpolar material for oral consumption. Examples of compositions also include a fluid for infusing a porous substrate.

As used herein, the terms “homogenous,” “substantially homogenous,” and their equivalents, can refer to a mixture of at least two ingredients that appears visibly mixed. One example of a homogenous solution is an emulsion, wherein a first fluid is arranged into droplets with diameters of between 1 nanometer and 1000 nanometers, and the droplets are suspended in a second fluid. In particular implementations, a homogenous mixture includes droplets of a first fluid suspended in a second fluid, wherein the droplets have diameters between 10 nanometers and 100 nanometers. The first fluid and the second fluid may not be miscible with each other. For instance, the first fluid may be polar and the second fluid may be nonpolar, or vice versa.

### Infused Beverages

Various implementations of the present disclosure can relate to homogenous mixtures of an aqueous solution and a nonpolar molecule. Examples of an aqueous solution include various brewed beverages, such as coffee and/or tea. As used herein, the terms “coffee,” “coffee beverage,” and their equivalents, can refer to water brewed with roasted beans of plants in the Coffea genus. Examples of plants that produce beans which can be used to brew coffee include Coffea arabica, Coffea canephora, or the like. Espresso is one example of coffee. As used herein, the terms “tea,” “tea beverage,” and their equivalents, can refer to water infused with vegetable matter. In some cases, the vegetable matter includes leaves of Camellia sinesis, and/or parts of other herbaceous plants (e.g., rooibos, mint, chamomile, or the like). Various brewed beverages described herein are emulsified with a nonpolar material, such as a cannabinoid, a nonpolar flavorant, nonpolar supplemental materials (e.g., vitamins, minerals, or other dietary supplements), or any combination thereof.

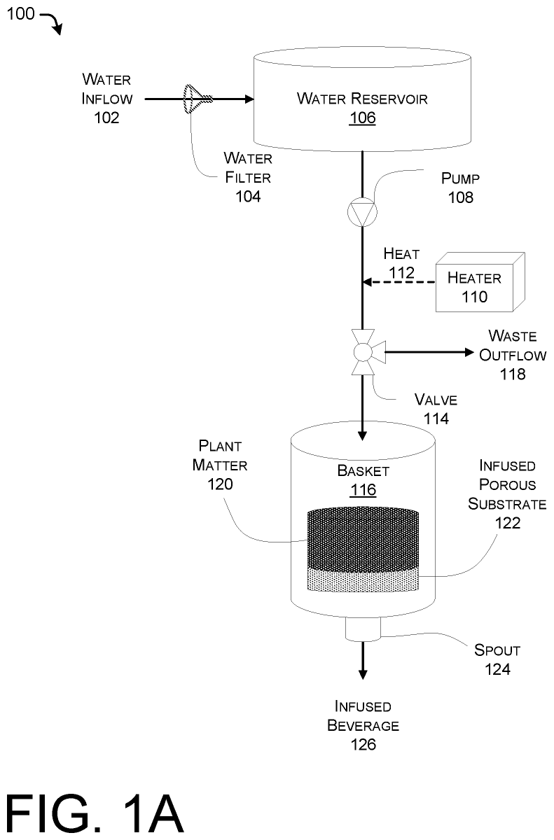

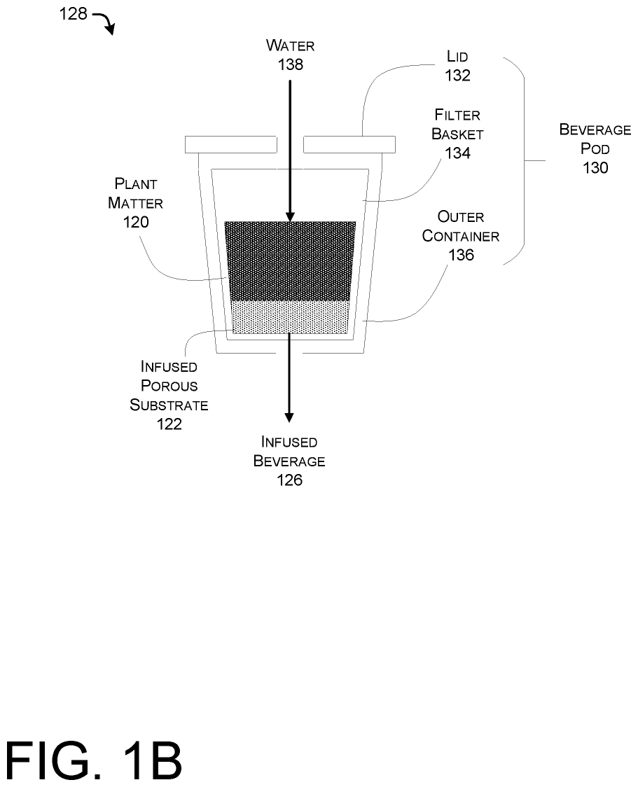

FIG. 1A illustrates an example system 100 for producing a homogenous mixture of a brewed beverage and a nonpolar material. As illustrated in FIG. 1A, the system 100 includes a water inflow 102, a water filter 104, a water reservoir 106, a pump 108, a heater 110, heat 112, a valve 114, a basket 116, waste outflow 118, plant matter 120, an infused porous substrate 122, and a spout 124 through which an infused beverage 126 outflows.

The system 100 can include at least one fluid circuit that includes one or more pipes, tubes, or the like that interconnect various elements in the system 100. The pipes, tubes, or the like may include a metal (e.g., copper, brass, stainless steel, etc.), a plastic (e.g., polyvinyl chloride, etc.), or the like. In various implementations, the fluid circuit can include materials that are food-safe at various temperatures.

In various implementations, various elements of the system 100 can be omitted without departing from the scope of the present disclosure. In some cases, at least one element (other than the infused filter 122) can be omitted from the system 100, according to some implementations. For instance, in some cases, the water inflow 102 or the water reservoir 106 may be omitted from the system 100. In some cases, at least one element can be arranged in a different order along the fluid circuit than the order depicted in FIG. 1A. For example, the heater 110 could be configured to apply heat to water flowing between the water reservoir 106 and the pump 108, to water flowing between the valve 114 and the basket 116, or the like.

The water inflow 102 can be used to input water into the fluid circuit. In some cases, the water inflow 102 can be a tap from an external reservoir, water faucet, or the like. The water filter 104 may filter water input by the water inflow 102. The water filter 104 may extract minerals, sediment, and other dissolved or suspended materials in the water that enters the fluid circuit from the water inflow 102. The dissolved and/or suspended materials may be removed from the water to prevent scale build-up within the fluid circuit, to improve taste of the final infused beverage 126, or the like. In various implementations, the water filter 104 may include at least one of a mechanical filter, a carbon filter, a scale inhibitor, an ion exchanger, a reverse osmosis system, or the like.

The water reservoir 106 may hold water filtered by the water filter 104. In some cases, the water reservoir 106 may be a tank that can at least temporarily hold water in the fluid circuit. The water reservoir 106, for instance, may include a metal (e.g., copper, brass, stainless steel, etc.), a plastic (e.g., polyvinyl chloride, etc.), or the like.

The pump 108 may pump water through the fluid circuit. For instance, the pump 108 may include at least one of a rotary pump, a vibration pump, or the like. The pump may pump water from the water reservoir 106 to the basket 116, in various implementations. In various examples, the pump may impose a water pressure inside of the fluid circuit, so that the water flows through the basket 116 at a substantial pressure (e.g., 9 bars). In some implementations, the pump 108 may be omitted, such that the water from the water reservoir 106 is propelled through the fluid circuit by hydrostatic pressure.

The heater 110 may apply heat 112 to the water in the fluid circuit. In various implementations, the heater 110 may apply the heat 112 to the water that has been pressurized by the pump 108. The heater 110 may include at least one of a thermoblock, a thermocoil, a boiler, or the like. In some cases, the heat 112 from the heater 110 may raise the temperature of the water to a threshold temperature between 160 to 210 degrees Fahrenheit (° F.) or 70 to 100 degrees Celsius (° C.). For example, the threshold temperature may be a threshold of 160° F., 170° F., 180° F., 190° F., 195° F., 200° F., 210° F., 70° C., 80° C., 90° C., 95° C., 100° C., or the like. For example, in instances in which the system 100 is used to generate infused tea, the threshold temperature may be 160° F. In examples in which the system 100 is used to generate coffee (e.g., Robusta coffee), the threshold temperature may be 210° F.

The valve 114 may control the flow of the heated water to a basket 116. For instance, the valve 114 may be a three-way valve. In some cases, the valve 114 may be an anti-vacuum valve. In various implementations, the valve 114 may be configured to selectively pass water to the basket 116 in that is at least a threshold water pressure. The threshold pressure may be between atmospheric pressure to 16 bars. For example, the threshold pressure may be 7 bars, 8 bars, 9 bars, 10 bars, 11 bars, 12 bars, 13 bars, 14 bars, or 16 bars. In some implementations, the threshold pressure may be at least 9 bars. In some examples in which the system 100 is used to generate infused espresso, the threshold pressure may be in a range of 8 bars to 16 bars. In some cases, the valve 114 may block the passage of water that is below the threshold water pressure. For instance, the pump 108 may be configured to increase the water pressure in the fluid circuit until the water is at least the threshold water pressure, and the valve 114 releases the pressurized water to the basket 116. According to some implementations, the valve 114 may include at least one of a solenoid valve, a group valve, a flow control valve, or the like.

In various implementations, the valve 114 may be a multi-way valve with a waste outflow 118. In some cases, the valve 114 may prevent backflow water from flowing from the basket 116 to the water reservoir 106 by siphoning the backflow out of the waste outflow 118. Accordingly, contamination of the water reservoir 106 with the plant matter 120 and/or water that has encountered the plant matter 120 may be prevented.

In various implementations, the basket 116 may receive the pressurized, heated water from the valve 114. As used herein, the term “basket” can refer to a structure at least partially enclosing a space, wherein the structure includes one or more openings (e.g., holes or pores) through which an aqueous solution can pass. In various implementations, the basket 116 can include a portafilter basket, a beverage pod (e.g., a K-CUP® pod from Keurig Dr Pepper Inc of Burlington, Mass., a NESPRESSO® capsule, from Nestlé Nespresso S.A. of Lausanne, Switzerland or the like), a brew basket (e.g., a coffee filter basket), a pour over coffee dripper (e.g., a CHEMEX® coffeemaker from Chemex Corporation of Chicopee, Mass., HARIO V60 from Hario USA, Inc. of Cerritos, Calif., or the like), or their equivalents. The basket 116 may include stainless steel, ceramic, glass (e.g., borosilicate glass), or any other material that is food safe and/or heat safe. The basket 116 may be configured to hold the plant matter 120 during a brewing process. Although not illustrated in FIG. 1A, the basket 116 may include an external handle. The basket 116 can include an outer wall surrounding an inner space. The outer wall can include at least one hole by which water can enter the basket 116 and/or an infused beverage can leave the basket 116. In various implementations, the outer wall includes multiple holes by which the infused beverage 126 can flow from the basket 116. For example, holes in a portafilter may have diameters in a range of 0.2 millimeters (mm) and 0.4 mm, such as a diameter of 0.2 mm, 0.3 mm, 0.4 mm, or some other distance. In some instances, the outer wall includes at least one porous screen including holes with widths and/or diameters in a range of 10 micrometers (um) to 400 um, such as a diameter of 10 um, 25 um, 50 um, 75 um, 100 um, 200 um, 300 um, 400 um, or some other distance.

The plant matter 120 may be disposed with the infused porous substrate 122 within the inner space of the basket 116. For example, the infused porous substrate 122 may be disposed between the plant matter 120 and a spout 124 of the basket 116, through which infused beverage 126 can be expelled from the basket 116. The plant matter 120 may include ground and/or roasted coffee beans (e.g., ground and/or roasted espresso beans), tea leaves (e.g., leaves of Camellia sinensis, or the like), herbaceous matter (e.g., flowers, leaves, roots, and/or stems of herbaceous plants, such as plants in the genus Mentha, plants in the genus Echinacea, Matricaria recutita, Chamaemelum nobile, Curcuma longa, or the like), and/or spices (e.g., cinnamon, cardamom, ginger, clove, peppercorn, or the like). As used herein, the term “coffee grounds” refers to ground coffee beans (e.g., ground espresso beans). In various implementations, the plant matter 120 (e.g., ground plant matter 120) can have a particle size (e.g., a radius or diameter) no more than 1.5 mm, such as a particle size of no more than 0.5 mm, 0.6 mm, 0.7 mm, 0.8 mm, 0.8 mm, 1.0 mm, 1.25 mm, or 1.5 mm.

In various examples, the plant matter 120 is tamped down in the inner space of the basket 116. For example, a blunt or flat instrument (e.g., an espresso tamper) is pressed onto the plant matter 120 disposed in the inner space of the basket 116. In some cases, the plant matter 120 is pressed at a pressure of 10 to 40 pounds (lbs) or 40 to 200 Newtons (N). According to various examples, tamping removes air between particles in the plant matter 120 and packs the particles in the plant matter 120 closely and consistently together. Thus, tamping can shape the plant matter 120 into an even puck through which the water entering the basket 116 can flow though evenly in order to generate the infused beverage 126.

In some implementations in which the plant matter 120 includes ground and/or roasted coffee beans, the infused beverage 126 can include infused coffee and/or espresso. In some cases in which the plant matter 120 includes tea leaves, herbaceous matter, and/or spices, the infused beverage 126 can include infused tea (e.g., black tea, green tea, white tea, red tea, herbal tea, or the like). The infused beverage 126 may be an example of a “composition,” as used herein.

In particular examples, the plant matter 120 is mixed with, or can be substituted for, one or more additional water-soluble materials. The water-soluble materials can be in the form of a powder (e.g., a powdered concentrate), an extract, a syrup (e.g., a sugar syrup), a liquid (e.g., a liquid concentrate), or a combination thereof. Examples of the water-soluble material include, for example: dried fruit extract or powder, liquid or powdered bouillon, powdered infant formula, powdered milk, powdered creamer, sweeteners, thickeners, flavorings, or the like.

In various implementations, the infused porous substrate 122 may include a porous material infused with a nonpolar material (e.g., a cannabinoid, a flavorant, a supplemental material, or the like). In some instances, the infused porous substrate 122 may include at least one of paper (e.g., cotton cellulose fiber filter paper, bleached and/or unbleached coffee filter paper, or the like), plant fiber (e.g., a cotton cellulose fiber filter, a wood cellulose fiber filter, or the like), a synthetic material (e.g., polyether sulfone (PES), or the like), a ceramic (e.g., aluminum oxide, zirconium oxide, graphite, or the like), a metal (e.g., stainless steel, copper, or the like) or some other type of porous material. In various implementations, the infused porous substrate 122 may include a food-safe porous material.

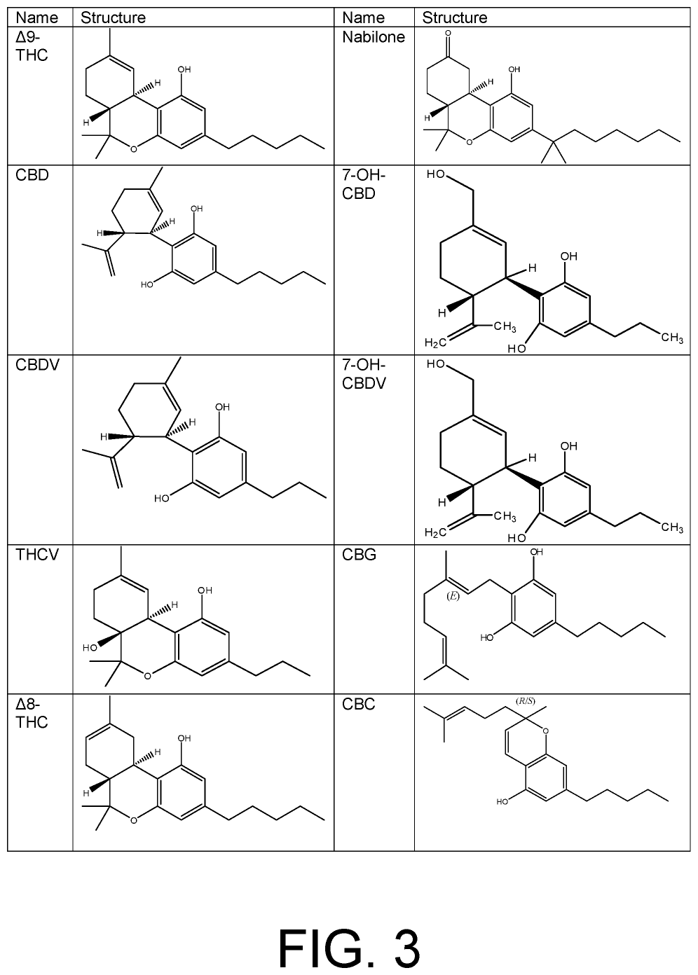

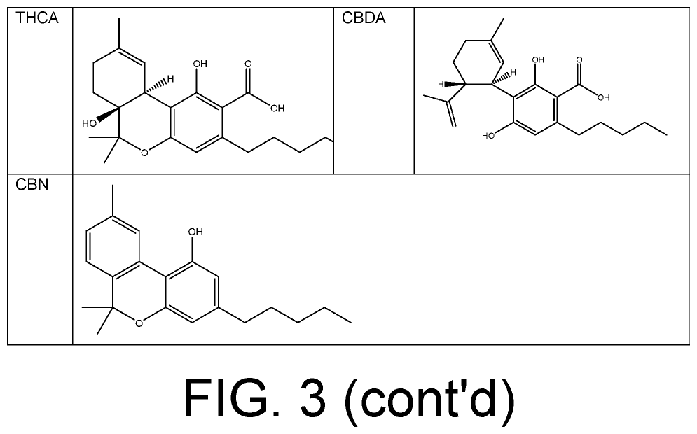

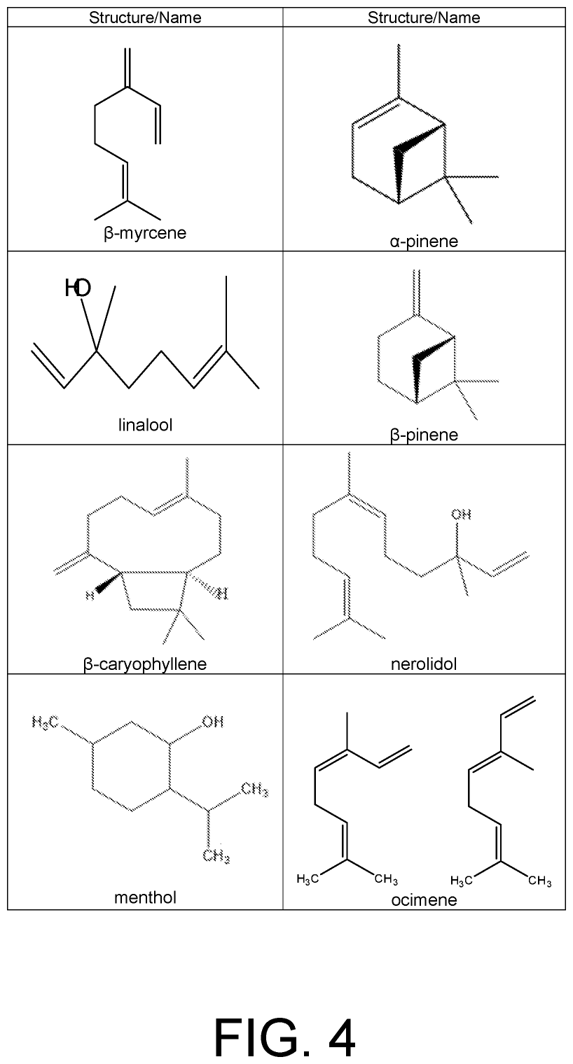

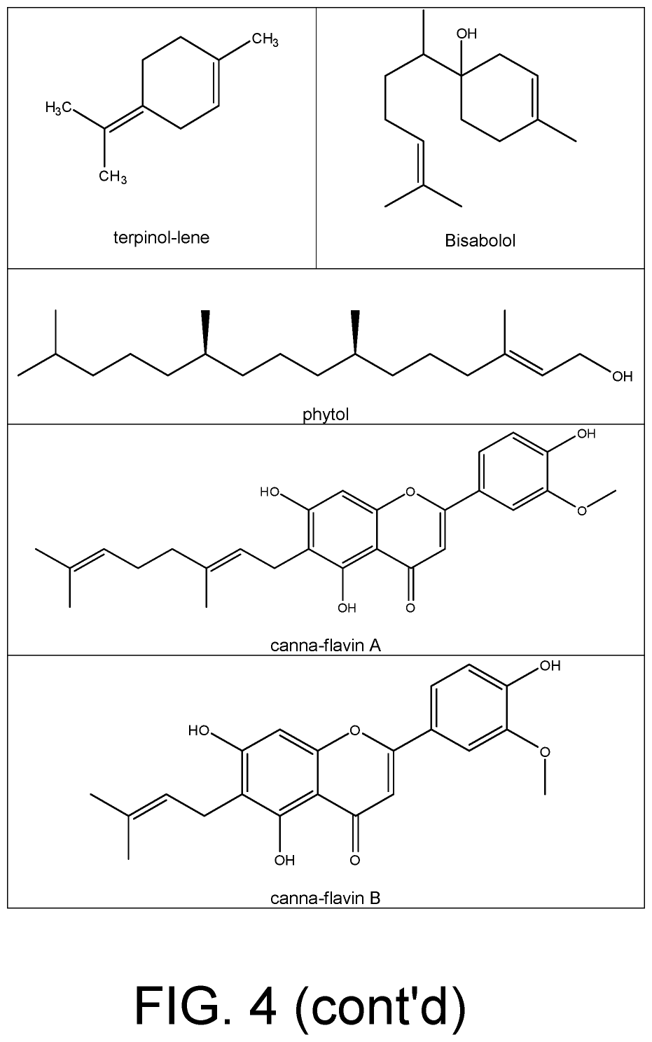

A solution containing the nonpolar material can be dried on and/or distributed in the infused porous substrate 122, for example. Various examples of nonpolar materials that can be distributed on and/or in the infused porous substrate 122 are described below, e.g., with reference to FIGS. 3 and 4. For example, cannabinoids such as THC and/or CBD can be distributed in the infused porous substrate 122. In various implementations, an amount of the cannabinoid(s) on or in the infused porous substrate 122 may be an amount sufficient to provide a threshold concentration and/or a threshold amount of the cannabinoid(s) in the infused beverage 126. The threshold concentration may be, for example, in a range of 0.001 mg/mL to 100 mg/mL, such as a concentration greater than or equal to 0.01 mg/mL and less than or equal to 100 mg/mL. For example, the threshold concentration may be a maximum concentration of 10 mg/mL. The threshold amount may be, for example, in a range of 1.0 mg to 10 grams (g). For example, the threshold amount can be greater than or equal to 2.0 mg and less than or equal to 50 mg. In various instances, the nonpolar material can include a flavorant, such as one or more aroma- and/or flavor-conferring molecules. In various examples, an amount of the flavorant on or in the infused porous substrate 122 may be an amount sufficient to provide a discernable aroma and/or flavor to a consumer of the infused beverage 126.

According to some implementations, at least one dispersal agent can be distributed on and/or in the infused porous substrate 122. Various examples of dispersal agents are described below. For instance, the dispersal agent(s) can include an amphipathic molecule. In various implementations, the dispersal agent(s) can increase the effective solubility of the nonpolar material in an aqueous solution, such as the infused beverage 126.

The infused porous substrate 122 may, in some cases, be infused with one or more additional ingredients, such as a supplemental material or an entourage-restoring molecule. In some cases, the infused porous substrate 122 is infused with one or more vitamins (e.g., vitamin D, vitamin E, or the like) or minerals (e.g., zinc, magnesium, or the like). In some cases, the infused porous substrate 122 is infused with turmeric and/or a curcuminoid (e.g., curcumin). The amount of the supplemental material in the infused porous substrate 122 may be an amount sufficient to provide a threshold concentration and/or a threshold amount of the supplemental material in the infused beverage 126. In various instances, the amount of vitamin A in the infused beverage 126 may be in a range of 700 ug to 32 mg; the amount of vitamin E in the infused beverage 126 may be in a range of 15 mg to 38 mg; the amount of one or more curcuminoids in the infused beverage 126 may be in a range of 200 mg to 6 g; and so on. The amount of the supplemental material in the infused porous substrate 122 may be incompletely transferred to the infused beverage 126. In some cases, the amount of vitamin A in the infused porous substrate 122 is in a range of 1.4 mg to 110 mg, the amount of vitamin E in the infused porous substrate 122 is in a range of 30 mg to 130 mg, the amount of the curcuminoid(s) in the infused porous substrate 122 is in a range of 400 mg to 20 g; and so on.

According to some examples, at least one terpene and/or flavonoid may be distributed and/or in the infused porous substrate 122. In some cases, the terpene(s) and/or flavonoid(s) may be at least a part of the nonpolar material transferred from the infused porous substrate 122 into the infused beverage 126. Examples of various terpenes and/or flavonoids that can be distributed in the infused porous substrate 122 are described below with reference to FIG. 4. Various terpenes and/or flavonoids may improve the effects of active ingredients, such as cannabinoids, when consumed with the active ingredients.

According to various examples of the present disclosure, the pressurized and heated water may be expressed through the plant matter 120 within the basket 116. A brewed solution (e.g., coffee, espresso, tea, or the like) may be generated as the water is expressed through the plant matter 120. In addition, the water may be expressed through the infused porous substrate 122 at the same or similar pressure. The pressure of the water (e.g., atmospheric pressure, 9 bars, or some other pressure) and/or the flow rate of the water expressed through the basket 116 and the infused porous substrate 122 can cause turbulent mixing of the nonpolar material within the infused porous substrate 122 and the brewed solution. The turbulent mixing enables the nonpolar material to be distributed into the brewed solution in a substantially homogenous mixture (e.g., an emulsion). Accordingly, despite the difference in polarity between the brewed solution, which is substantially polar, and the nonpolar material, the nonpolar material can be effectively distributed throughout the resultant infused beverage 126. That is, a separated layer (e.g., an oily layer) of the nonpolar material may be absent from the infused beverage 126, thereby improving the taste and/or mouthfeel of the infused beverage 126 for individuals consuming the infused beverage 126. Furthermore, in some cases, the homogenous mixture can be generated without additional additives (e.g., dispersal agents).

In addition, other materials on and/or within the infused porous substrate 122 can be mixed into the infused beverage 126. In some cases, a supplemental material (e.g., vitamin D, vitamin E, turmeric, a curcuminoid, or the like) is transferred from the infused porous substrate 122 to the infused beverage 126. In some instances, a flavorant in and/or on the infused porous substrate 122 can impart a discernable flavor and/or aroma in the infused beverage 126. Further, in some cases in which entourage-restoring molecules, such as terpenes and/or flavonoids, are included in the infused porous substrate 122, the entourage-restoring molecules may further enhance the effects of the active ingredient(s) on drinkers of the infused beverage 126.

In some cases, the infused beverage 126 can be consumed directly by a subject (e.g., a human). In various implementations, the infused beverage 126 can be included in another orally consumable product, such as a food item. For instance, the infused beverage 126 can be included in ice cream, cakes, cookies, or the like. In some cases, the infused beverage 126 can be added to another material to produce a coffee-based beverage, the material including at least one of nitrogen gas, a carbonated liquid, steam, a dairy product, a dairy substitute, a sugar, a syrup, or an alcohol.

FIG. 1B is a cross-sectional diagram illustrating an example system 128 for producing a homogenous mixture of coffee and a nonpolar material using a beverage pod 130. As illustrated, the beverage pod 130 can include a lid 132, an outer container 134, and a filter basket 136. As used herein, the term “filter basket” refers to a basket that includes a porous material. Using the beverage pod illustrated in FIG. 1B, water 138 can be converted into infused beverage 126. In various implementations, the beverage pod 130 can be used as the basket 116 described above with reference to FIG. 1A. According to some implementations, the beverage pod 130 may be reusable. In some cases, the beverage pod 130 can be disposable.

The lid 132 may at least partially cover an opening in the outer container 136. The lid 132 can include a hole that exposes an inner space of the beverage pod 130 to a space outside of the beverage pod 130. In various implementations, the hole may be premanufactured in the lid 132. According to some examples, the lid 132 may include a pierceable material and the hole can be generated by piercing the lid 132 with a needle (not illustrated). For example, the lid 132 may include at least one of a metal (e.g., aluminum), a polymer, or a laminate material (e.g., a metallic/polymer laminate). The lid 132 may be disposed at least partially over an opening of the outer container 136. In some implementations, the lid 132 is attached to a rim of the outer container 136 by an adhesive, thermal bonding, chemical bonding, or the like. According to some examples, an outer edge portion of the lid 132 is crimped around the rim of the outer container 136. According to various implementations, the lid 132 can be mechanically attached to the outer container 136. For example, the lid 132 may be a screw-top lid that can be screwed onto the rim of the outer container 136. In some instances, the lid 132 can be omitted from the beverage pod 130.

The outer container 136 includes one or more sidewalls and the rim, which may be attached to the lid 132. In various implementations, the outer container 136 is cup-shaped. In particular cases, the sidewall(s) extend at a nonparallel (e.g., a non-180-degree angle, such as an angle greater than 0 degrees and less than 180 degrees) angle from a bottom wall. In some examples, the outer container 136 has a curved sidewall and/or bottom wall. In some cases, the bottom wall is substantially disk-shaped and/or the rim is substantially circular. In various cases, at least one surface of the outer container 136 is concave in at least two planes (e.g., concave in an xy plane and an xz plane). As illustrated in FIG. 1B, the sidewall(s) of the outer container 136 may be tapered, such that a diameter of the bottom wall of the outer container 136 is smaller than a diameter of the rim of the outer container 136. The outer container 136 may include one or more solid materials that are impervious to water, aqueous solutions, and/or gasses. For example, the outer container 136 may include at least one of polyethylene, ethylene vinyl alcohol (EVOH), or polystyrene. The outer container 136 may have a hole through which water and/or an aqueous solution can flow. For example, the hole may be through the bottom wall of the outer container 136. In some cases, the hole may be pre-manufactured in the outer container 136. In some cases, the outer container 136 may include a pierceable material and the hole can be generated by piercing the outer container 136 with a needle (not illustrated), such as a needle used to generate the hole in the lid 132.

The filter basket 134 may be disposed inside of a space defined by the lid 132 and/or the outer container 136. As used herein, the term “filter” can refer to any porous material that is configured to pass fluids and/or particles that are smaller than a threshold size (e.g., particles with widths that are smaller than widths of pores in the filter) and to retain particles that are greater than or equal to the threshold size (e.g., particles with widths that are larger than the widths of the pores). In various implementations, the filter basket 134 may be cone- or cup-shaped. The filter basket 134 may be smaller than the outer container 136, such that the filter basket 134 can be spaced apart from the walls of the outer container 136 when the filter basket 134 is disposed inside of the space defined (e.g., at least partially enclosed) by the outer container 136. A rim of the filter basket 134 may be attached to the sidewall(s) and/or the rim of the outer container 136. The filter basket 134 may be permeable to water, an aqueous solution, and/or gasses. In various implementations, the filter basket 134 may include at least one of paper, pulp, a polymer (e.g., polypropylene), cellulose, a metal (e.g., stainless steel, copper, etc.), or the like. In some implementations, the filter basket 134 may include pores with diameters that are no larger than particle sizes (e.g., diameters) of particles in the plant matter 120. For example, the filter basket 134 may include pores with widths that are no larger than 50 nm, 0.1 mm, 0.5 mm, 0.6 mm, 0.7 mm, 0.8 mm, 0.9 mm, or 1.0 mm. The plant matter 120 and the infused porous substrate 122 may be disposed in an inner space defined by and/or at least partially enclosed by the filter basket 134. Although the infused porous substrate 122 is illustrated in FIG. 1B underneath the plant matter 120, implementations are not limited to this physical arrangement. For example, the porous substrate 122 may be disposed inside of the plant matter 120 and/or disposed on top of the plant matter 120.

In various implementations, the water 138 may enter the beverage pod 130 through the hole in the lid 132. In some cases, the water 138 may be pre-heated. The water 138 may be exposed to the plant matter 120 in the filter basket 134 and may therefore be infused with the plant matter 120, thereby generating a brewed beverage, such as coffee, espresso, and/or tea. In various examples, a pressure differential may exist between the inner space of the filter basket 134 and the space between the filter basket 134 and the outer container 136. Accordingly, the water may flow from the inside of the filter basket 134 to the space between the filter basket 134 and the outer container 136. In various implementations, the water may flow through the infused porous substrate 122. For instance, the water may turbulently flow through the infused porous substrate 122, due to the sizes of pores in the porous substrate 122, the flow rate of water in the beverage pod 130, and/or the magnitude of a pressure differential associated with the beverage pod 130. Accordingly, the water may be infused with various materials in and/or on the infused porous substrate 122, such as one or more nonpolar material (e.g., a cannabinoid, a flavorant, a supplemental material, or the like), dispersal agents, and/or entourage-restoring molecules. The infused beverage 126 may include the water 138 infused with the plant matter 120 and materials disposed in and/or on the infused porous substrate 122. In various cases, a pressure differential may exist at the hole in the outer container 136, such that the infused beverage 126 may flow from the space between the filter basket and the outer container 136 through the hole in the outer container 136. The infused beverage 126 may include a substantially homogenous mixture (e.g., an emulsion) of the brewed beverage and the nonpolar material.

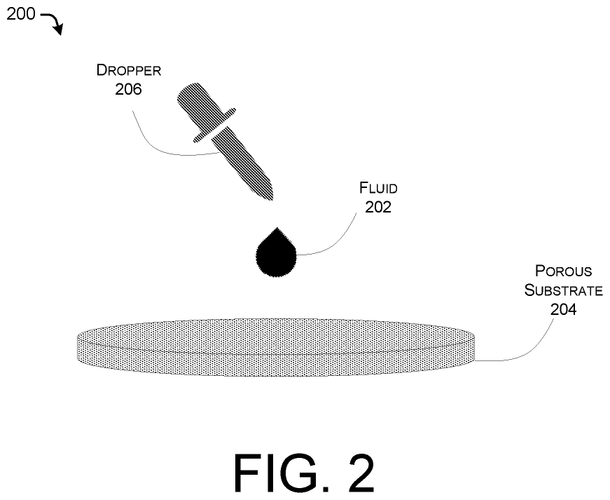

FIG. 2 illustrates an example environment 200 for generating an infused porous substrate for producing homogenous brewed beverages including nonpolar materials. For instance, the environment 200 can be used to generate the infused porous substrate 122 described above with reference to FIGS. 1A and 1B.

As illustrated in FIG. 2, a fluid 202 containing a nonpolar material can be distributed onto a porous substrate 204. In some cases, the nonpolar material can include a cannabinoid, a flavorant, a supplemental material, and/or an entourage-restoring molecule. In some cases, the fluid 202 may include a tincture, an oil, or the like. The fluid 202 may be an example of a “composition,” as used herein. For instance, the fluid 202 may include the nonpolar material suspended in a solvent that is nonpolar, amphipathic, or a combination thereof. The solvent may include, for instance, an alcohol (e.g., ethanol and/or an aqueous solution of ethanol), butters, plant-based oils (e.g., coconut oil, grape seed oil, hazelnut oil, olive oil, palm oil, papaya seed oil, peanut oil, sesame oil, sprouted wheat oil, wheat germ oil, etc.), or any combination thereof. Examples of cannabinoids that can be included in the fluid 202 as nonpolar material are described below with reference to FIG. 3. In some cases, the fluid 202 can include a supplemental material, such as vitamin A, vitamin E, turmeric, and/or a curcuminoid. In various implementations, the fluid 202 can include at least one terpene and/or flavonoid, examples of which are described below with reference to FIG. 4.

In various implementations, a dropper 206 can be used to distribute a particular volume of the fluid 202 onto the porous substrate 204. In some implementations, the particular volume can be between 0.1 milliliters (mL) and 3 mL. For instance, the particular volume can be 0.1 mL, 0.5 mL, 1.0 mL, 1.5 mL, 2.0 mL, 2.5 mL, 3.0 mL, or some other volume.

In various implementations, the fluid 202 includes an amount of a material (e.g., the nonpolar material) sufficient to generate a desired amount of the material in a substantially homogenous mixture of an aqueous solution prepared using the porous substrate 204. The desired amount of the material in the substantially homogenous mixture includes, for instance, at least an effective amount of the material. In some examples, the fluid 202 includes 30% to 50% of the desired amount (e.g., mass) of the material to be infused in the substantially homogenous mixture. The desired amount, for instance, is an effective amount of the material. For example, the desired amount of the cannabinoid(s) and/or entourage-restoring molecule(s) in the substantially homogenous mixture may be an amount of the cannabinoid(s) and/or entourage-restoring molecule(s) that can produce a desired effect (e.g., a recreational effect) when the substantially homogenous mixture is consumed orally by an individual. For example, the desired amount of the cannabinoid(s) and/or entourage-restoring molecule(s) may be in a range of 1 mg to 10 g, such that the amount of the cannabinoid(s) and/or entourage-restoring molecule(s) in the fluid 202 is in a range of 2.0 mg to 30 g. In some examples, the desired amount of the flavorant(s) in the substantially homogenous mixture may be an amount of the flavorant(s) that can produce a desired taste when the substantially homogenous mixture is consumed orally by an individual. For instance, the desired amount of the flavorant(s) in the substantially homogenous mixture may be in a range of 0.1 ug to 6 g, such that the amount of the flavorant(s) in the fluid 202 is in a range of 0.2 ug to 20 g. In some examples, the desired amount of supplemental material(s) in the substantially homogenous mixture may be an amount sufficient to satisfy a daily dietary allowance of the supplemental material(s) when the substantially homogenous mixture is consumed orally by an individual, such as an allowance recommended by the U.S. National Institutes of Health, Office of Dietary Supplements. In various instances, the desired amount of vitamin A in the substantially homogenous mixture may be in a range of 700 ug to 32 mg, such that the amount of vitamin A in the fluid 202 is in a range of 1.4 mg to 110 mg; the desired amount of vitamin E in the substantially homogenous mixture may be in a range of 15 mg to 38 mg, such that the amount of vitamin E in the fluid 202 is in a range of 30 mg to 130 mg; the desired amount of one or more curcuminoids in the substantially homogenous mixture may be in a range of 200 mg to 6 g, such that the amount of the curcuminoid(s) in the fluid 202 is in a range of 400 mg to 20 g; and so on.

In various implementations, the particular volume of the fluid 202 may propagate substantially throughout the porous substrate 204 via capillary action. For instance, the porous substrate 204 may be a porous structure including, for instance, a paper filter (e.g., cotton cellulose fiber filter paper, bleached and/or unbleached coffee filter paper, or the like), a fiber filter (e.g., a cotton cellulose fiber filter, a wood cellulose fiber filter, or the like), a synthetic filter (e.g., a polyether sulfone (PES) filter) or some other type of filter. The sizes of the pores within the porous substrate 204 may be relatively small. Accordingly, surface tension of the fluid 202 and/or adhesive forces between the fluid 202 and the walls of the pores of the porous substrate 204 may propel the fluid 202 through the pores via capillary action. As a result, the fluid 202 can spontaneously propagate throughout the porous substrate 204.

An infused porous substrate may include the porous substrate 204 and the fluid 202, which may be distributed throughout the porous substrate 204. In some cases, the infused porous substrate may be disposed in a basket (e.g., the basket 116) and used to produce the substantially homogenous mixture. For instance, the infused porous substrate may be used to produce an infused beverage (e.g., cannabinoid-infused espresso).

### Cannabinoids

In some implementations, compositions include mixtures of one or more cannabinoids. The one or more cannabinoids may be produced naturally and/or synthetically.

Various compositions described herein may include one or more primary cannabinoids. In particular implementations, primary cannabinoids are cannabinoids that exert the primary desired physiological effects of cannabis. Examples of cannabinoids that exert primary desired physiological effects of cannabis include tetrahydrocannabinol (THC) (e.g., Δ9-THC, Δ8-THC, or the like) and/or CBD. Primary cannabinoids can also include derivatives and/or analogs of a cannabinoid that exert a primary desired physiological effect of cannabis.

The term “derivative” refers to a compound that is obtained from a similar compound or a precursor compound by a chemical reaction. The term “analog” (also “structural analog” or “chemical analog”) refers to a compound that is structurally similar to another compound but differs with respect to certain components, such as an atom, a functional group, and/or a substructure. Examples of analogs of THC include nabilone, ajulemic acid, and (−) HU-210.

In particular implementations, the primary cannabinoids include THC. THC is the predominant cannabinoid present in many cannabis strains, at often 10-20% of the dry weight of cannabis flowers. THC content in cannabis can vary from trace amounts (<1%) to over 30%. Many THC dominant cannabis strains contain only trace amounts of cannabidiol (CBD) (<1%). An example of a THC-dominant, low-CBD cannabis strain is Sour Diesel (22% THC). Cannabis and/or cannabis extracts containing THC (>1%) can be useful to provide a physiological and/or medical benefit of THC. An exemplary structure of THC is shown in FIG. 3.

In particular implementations, formulations can include CBD. In certain cannabis strains, such as Charlotte's Web™ (Stanley Brothers Social Enterprises, LLC, Colorado Springs, Colo.), CBD is the predominant cannabinoid. Charlotte's Web™ contains an average of 20% CBD and trace amounts of THC (0.3%), as measured by dry weight in cannabis flowers. CBD-dominant (>1%), low-THC (<1%) cannabis strains can be used for medicinal and nutritional benefits, and can be desirable in certain situations because they lack the psychoactive effects of THC. CBD content in cannabis can range from trace amounts (<1%) to over 20%. An exemplary structure of CBD is shown in FIG. 3.

In particular implementations, compositions include a combination of THC and CBD. Examples of cannabis strains that contain THC and CBD (over 1% each) include Harlequin, (5% THC and 12% CBD) and CBD Mango Haze (14% THC and 16% CBD). The health benefits of THC and CBD can be enhanced when the two molecules are provided together. For example, a combination of THC and CBD is thought to optimize certain analgesic and anxiolytic properties of the two cannabinoids. Furthermore, CBD can reduce or eliminate negative side effects of THC. The ratio of THC:CBD in cannabis strains can range from >100:1 THC:CBD to <0.01:1 THC:CBD.

In particular implementations, the primary cannabinoids include nabilone. Nabilone is a synthetic THC analog that is used for anxiolytic and antiemetic properties, and is also useful for treating pain of various etiologies such as multiple sclerosis (MS), peripheral neuropathy and spinal injuries (Lancet, 1995, 345, 579, Pharm. J. 259, 104, 1997; Baker & Pryce, Expert Opin Investig Drugs. 2003 April; 12(4):561-7). Nabilone is commonly administered in 1-2 mg doses, up to 6 mg per day. An exemplary structure of nabilone is shown in FIG. 3.

In particular implementations, formulations include cannabinoids that are synthetically produced. Examples of techniques for synthetic production of cannabinoids can be found in US2016/0355853; JP2016/509842; Petrzilka et al., Helv Chim Acta. 1967. 50(2):719-723; Kobayashi et al., Org Lett. 2006. 8(13):2699-2702; and Mechoulam & Gaoni, J Am Chem Soc. 1965. 87(14):3273-3275.

In particular implementations, one or more decarboxylated cannabis extracts are included in the compositions to provide primary cannabinoids. Decarboxylated cannabis extracts containing THC and/or CBD can be commercially available from sources including BioCBD+, Active CBD oil, RSHO™ (Medical Marijuana, Inc., Poway, Calif.), and Ethos Innovates™ (One LED Corp, Bainbridge Island, Wash.). Commercially available THC cannabis extracts include Zoots™ (Natural Extractions, LLC, University Place, Wash.); Dixie Elixirs, Marijuana Drops (Marijuana Market), and Ethos Innovates.

In particular implementations, primary cannabinoids can include non-decarboxylated cannabinoids, such as tetrahydrocannabinolic acid (THCA) and/or cannabidiolic acid (CBDA). For example, primary cannabinoids can be purchased as non-decarboxylated cannabis extracts (containing THCA instead of THC and/or CBDA instead of CBD), and can be decarboxylated during formulation (e.g., brew, extraction, and/or generation of a composition). The relative cannabinoid content of a cannabis strain is typically preserved during extraction (e.g. CO₂ or BHO extraction). Extracts sourced from a single strain can be useful for mimicking the entourage effects of a particular strain by providing the strain's natural repertoire of primary and additional cannabinoids. Non-decarboxylated cannabis extracts are commonly available for a wide variety of cannabis strains, such as Sour Diesel, Super Lemon Haze, Pure Kush, Charlotte's Web™ and Durban Poison.

In particular implementations, cannabinoids that are provided in non-decarboxylated cannabis extracts are decarboxylated prior to formulating compositions for oral delivery. Decarboxylation of cannabinoids in a cannabis extract can be performed by heating the cannabis extract in a boiling water bath for 90 minutes. In particular implementations, decarboxylation of the cannabinoids is performed prior to mixing with entourage-restoring molecules, because certain entourage-restoring molecules can be destroyed by heat.

According to various implementations of the present disclosure, compositions and/or formulations may include one or more natural and/or synthetic cannabinoids as the primary cannabinoids. Examples of cannabinoids derived from plants include cannabigerol (CBG), cannabichromene (CBC), cannabinol (CBN), CBD, THC, iso-THC, cannabielsoin (CBE), cannabicyclol (CBL), cannabidivarin (CBDV), tetrahydrocannabivarin (THCV), CBDA, THCA, and cannabicitran (CBT). In particular implementations, synthetic cannabinoids include natural cannabinoids that are synthesized chemically and also their analogs and derivatives. Derivatives of natural cannabinoids can include metabolites of cannabinoids which are disclosed in WO 2015/198078. For example, the metabolite of CBD includes 7-OH-CBD and the metabolite of CBDV includes 7-OH-CBDV.

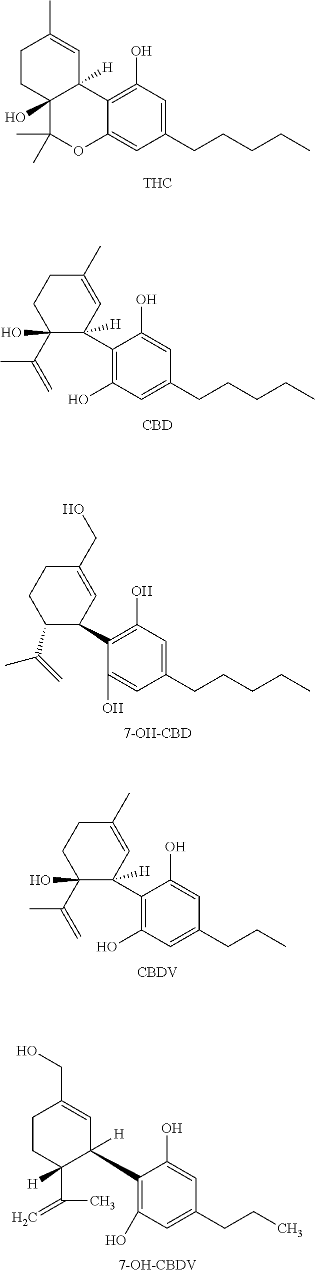

Other examples of synthetic cannabinoids include 3-carbamoyl-2-pyridone, and its derivatives and/or analogs disclosed in US 2008/0103139; pyrimidine derivatives and/or analogs disclosed in US 2006/0293354; carenadiol and its derivatives and/or analogs thereof disclosed in U.S. Pat. No. 4,758,597; cannabinoid carboxylic acids and their derivatives and/or analogs disclosed in WO 2013/045115; pyrido[3,2-E][1,2,4]triazolo[4,3-C]pyrimidine and its derivatives and/or analogs disclosed in WO 2008/118414; tetrahydro-pyrazolo[3,4-C] pyridine and its derivatives and/or analogs disclosed in WO 2007/112399; bicyclo[3.1.1]heptan-2-one cannabinoid and its derivatives and/or analogs disclosed in WO 2006/043260; resorcinol and its derivatives and/or analogs disclosed in WO 2005/0123051; dexanabinol compounds and their derivatives and/or analogs disclosed in WO 2004/050011; cannabimimetic lipid amide compounds and their derivatives and/or analogs disclosed in WO 2000/032200; nabilone and its derivatives and/or analogs disclosed in US 2010/0168066; 2-oxoquinolone compounds and their derivatives and/or analogs disclosed in US 2003/0191069; and 3,4-diaryl-4,5-dihydro-(h)-pyrazole-1-carboxamide and its derivatives and/or analogs disclosed in US 2011/0137040.

In particular implementations, 3-carbamoyl-2-pyridone and its derivatives and/or analogs include methyl 3-methyl-2-{[2-oxo-1-(2-oxo-ethyl)-1,2,5,6,7,8,9,10-octahydro-cycloocta[b]pyridine-3-carbonyl]-amino}-butyrate; dimethyl 2-[(1-cyclohexylmethyl-5,6-dimethyl-2-oxo-1,2-dihydropyridine-3-carbonyl)-amino]-succinate; and methyl 2-{[1-(3-methoxycarbonyamino-propyl)-2-oxo-1,2,5,6,7,8,9,10-octahydro-cycloocta[b]pyridine-3-carbonyl]-amino}-2-methyl-propionate.

In particular implementations, pyrimidine derivatives and/or analogs include a compound having Formula (I) (2-((2,4-dichlorophenyl)amino)-N-((tetrahydro-2H-pyran-4-yl)methyl)-4-(trifluoromethyl)pyrimidine-5-carboxamide),

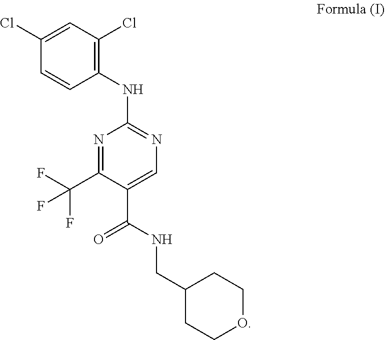 [SMILES: O=C(NCC1CCOCC1)c1cnc(Nc2ccc(Cl)cc2Cl)nc1C(F)(F)F]

Other pyrimidine derivatives and/or analogs include 2-(3-Chlorophenylamino)-4-trifluoromethylpyrimidine-5-carboxylic acid cyclohexylmethyl-amide; 2-Phenylamino-4-trifluoromethylpyrimidine-5-carboxylic acid cyclohexylmethyl-amide; 1-[2-(2,3-Dichlorophenylamino)-4-trifluoromethylpyrimidin-5-yl]-1-morphol-in-4-yl-methanone; 1-[2-(2,4-Dichlorophenylamino)-4-trifluoromethylpyrimidin-5-yl]-1-morphol-in-4-yl-methanone; and 2-(3-Chlorophenylamino)-4-trifluoromethylpyrimidin-5-carboxylic acid cyclopentylamide.

In particular implementations, carenadiol and its derivatives and/or analogs include compounds having Formula (II),

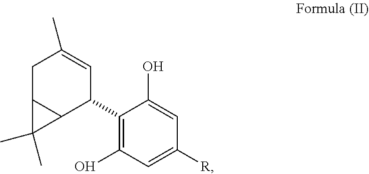

wherein R is a lower alkyl having 1 to 9 carbon atoms including isomeric forms such as i-butyl, n-butyl, and t-butyl. In particular implementations, R is C₅H₁₁ or 1,1-dimethylheptyl.

In particular implementations, cannabinoid carboxylic acids and their derivatives and/or analogs include compounds having Formula (III), (IV), (V), or (VI),

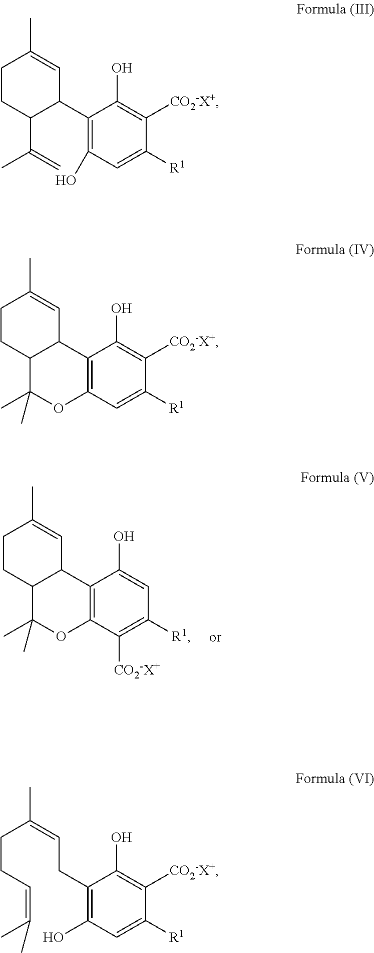

wherein:
R¹ is a straight-chain, branched or cyclic hydrocarbon residue with one C atom to 12 C atoms; and
X⁺ is NH₄⁺, mono-, di- or trivalent metal ions; or primary, secondary, tertiary or quaternary organic ammonium ions with up to 48 C atoms, which may bear still further functional groups.

Examples of multivalent ammonium ions include N,N-dicyclo-hexylamine-H+ and N,N-dicyclohexyl-N-ethylamine-H+. X+ can also be the hydrogen cation of a pharmaceutical active substance with at least one basic nitrogen atom, such as for example morphine, methadone (or an enantiomer thereof) or hydromorphone.

In particular implementations, pyrido[3,2-E][1,2,4]triazolo[4,3-C]pyrimidine and its derivatives and/or analogs include 5-tert-butyl-8-(2-chlorophenyl)-9-(4-chlorophenyl)pyrido[3,2-e][1,2,4]triazolo[4,3-c]pyrimidin-3(2H)-one; 8-(4-bromo-2-chlorophenyl)-5-tert-butyl-9-(4-chlorophenyl)pyrido[3,2-e][1,2,4]triazolo[4,3-c]pyrimidin-3(2H)-one; 5-fert-butyl-9-(4-chlorophenyl)-8-(2-methylphenyl)pyrido[3,2-e][1,2,4]triazolo[4,3-c]pyrimidin-3(2H)-one; 9-(4-bromophenyl)-5-tert-butyl-8-(2-chlorophenyl)pyrido[3,2-e][1,2,4]triazolo[4,3-c]pyrimidin-3(2H)-one; and 5-tert-butyl-8-(2-chlorophenyl)-9-(4-chlorophenyl)pyrido[3,2-e][1,2,4]triazolo[4,3-c]pyrimidine.

In particular implementations, tetrahydro-pyrazolo[3,4-C] pyridine and its analogs and/or derivatives include compounds having Formula (VII), (VIII), (IX), (X), or (XI),

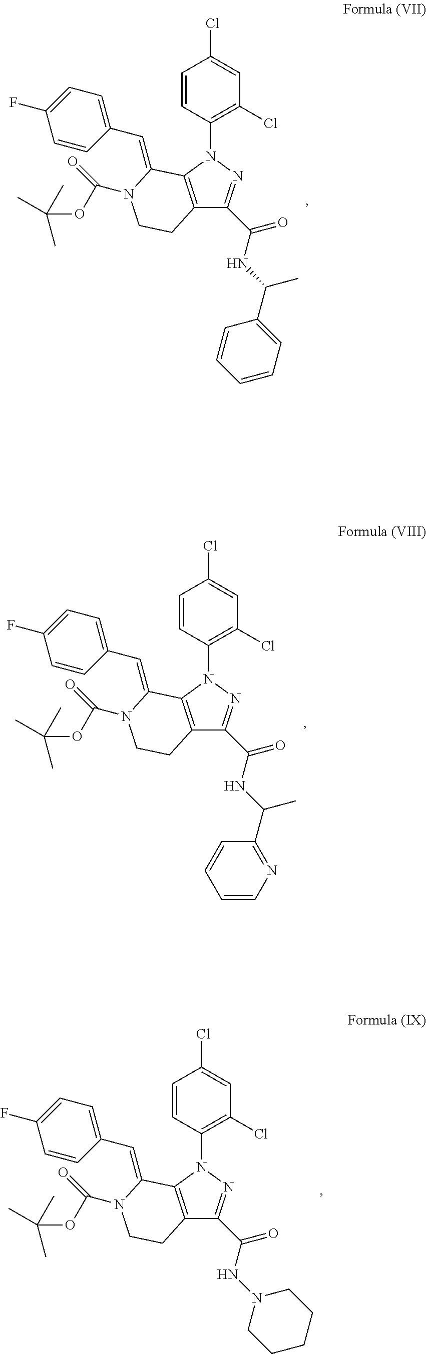 [SMILES: C[C@@H](NC(=O)c1nn(-c2ccc(Cl)cc2Cl)c2c1CCN(C(=O)OC(C)(C)C)/C2=C\c1ccc(F)cc1)c1ccccc1 ; CC(NC(=O)c1nn(-c2ccc(Cl)cc2Cl)c2c1CCN(C(=O)OC(C)(C)C)/C2=C\c1ccc(F)cc1)c1ccccn1 ; CC(C)(C)OC(=O)N1CCc2c(C(=O)NN3CCCCC3)nn(-c3ccc(Cl)cc3Cl)c2/C1=C/c1ccc(F)cc1]

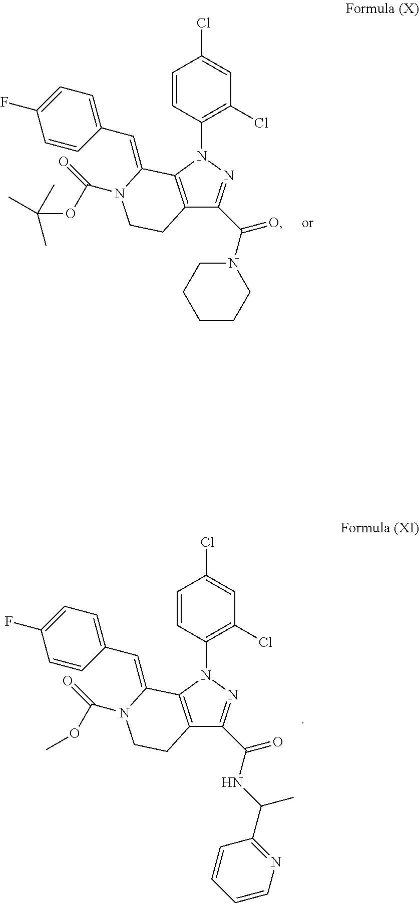 [SMILES: CC(C)(C)OC(=O)N1CCc2c(C(=O)N3CCCCC3)nn(-c3ccc(Cl)cc3Cl)c2/C1=C/c1ccc(F)cc1 ; COC(=O)N1CCc2c(C(=O)NC(C)c3ccccn3)nn(-c3ccc(Cl)cc3Cl)c2/C1=C/c1ccc(F)cc1]

In particular implementations, bicyclo[3.1.1]heptan-2-one cannabinoids and their derivatives and/or analogs include compounds having Formula (XII),

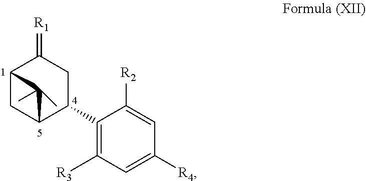

having a specific stereochemistry wherein C-4 is S, the protons at C-1 and C-5 are cis in relation to one another and the protons at C-4 and C-5 are trans; and wherein:
R₁ is (a) O or S; (b) C(R′)₂ wherein R′ at each occurrence is independently selected from the group consisting of hydrogen, cyano, —OR″, —N(R″)₂, a saturated or unsaturated, linear or branched C₁-C₆ alkyl, C₁-C₆ alkyl-OR″ or C₁-C₆alkyl-N(R″)₂ wherein at each occurrence R″ is independently selected from the group consisting of hydrogen, C(O)R′″, C(O)N(R′″)₂, C(S)R′″, saturated or unsaturated, linear or branched C₁-C₆ alkyl, C₁-C₆ alkyl-OR′″, and C₁-C₆ alkyl-N(R′″)₂, wherein at each occurrence R″′ is independently selected from the group consisting of hydrogen or saturated or unsaturated, linear, branched or cyclic C₁-C₁₂alkyl; or (c) NR″ or N—OR″ wherein R″ is as previously defined;
R₂ and R₃ are each independently (a) —R″, —OR″, —N(R″)₂, —SR″, —S(O)(0)NR″, wherein at each occurrence R″ is as previously defined; (b) —S(O)R^{b}, —S(O)(O)R^{b} wherein R^{b} is selected from the group consisting of hydrogen, saturated or unsaturated, linear or branched C₁-C₆alkyl, C₁-C₆alkyl-OR″, and C₁-C₆alkyl-N(R″)₂, wherein R″ is as previously defined; or (c) —OC(O)OH, —OS(O)(O)OR^{e}, —OP(O)(OR^{e})₂, —OR^{d} or —OC(O)—R^{d} chain terminated by —C(O)OH, —S(O)(O)OR^{e}, or —P(O)(OR^{e})₂, wherein R^{d} is a saturated or unsaturated, linear or branched C₁-C₆ alkyl and R^{e} is at each occurrence selected from the group consisting of hydrogen and R^{d} as previously defined; and
R₄ is (a) R wherein R is selected from the group consisting of hydrogen, halogen, OR′″, OC(O)R′″, C(O)OR′″, C(O)R′″, OC(O)OR′″, CN, N(R′)₂, NC(O)R′″, NC(O)OR′″, C(O)N(R′″)₂, NC(O)N(R′″)₂, and SR′″, wherein at each occurrence R′″ is as previously defined; (b) a saturated or unsaturated, linear, branched or cyclic C₁-C₁₂ alkyl-R wherein R is as previously defined; (c) an aromatic ring which can be further substituted at any position by R wherein R is as previously defined; or (d) a saturated or unsaturated, linear, branched or cyclic C₁-C₁₂ alkyl optionally terminated by an aromatic ring which can be further substituted as defined in (c).

In particular implementations, resorcinol and its derivatives and/or analogs include compounds having Formula (XIII),

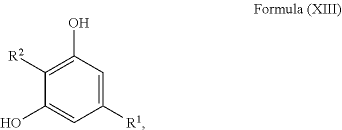

wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid including from 10 to 30 carbon atoms.

In particular implementations, resorcinol and its derivatives and/or analogs include compounds having Formula (XIII), wherein R1 and R2 are as follows:
R¹ is a straight alkyl chain of 5 to 8 carbon atoms, optionally substituted with one methyl group; and
R² is selected from geranyl optionally substituted with one —OH, and farnesyl optionally substituted with one —OH.

In particular implementations, resorcinol and its derivatives and/or analogs include compounds having Formula (XIII), wherein:
R¹ is (a) straight or branched alkyl chain of 7 to 12 carbon atoms; (b) —O—R³, where R³ is a straight or branched alkyl chain of 5 to 9 carbon atoms, optionally substituted by one phenyl group; or (c) —(CH₂)ₙ—O—R⁴, where n is an integer from 1 to 7, and R⁴ is a straight alkyl chain of 1 to 5 carbon atoms; and
R² is a non-cyclic terpenoid including from 10 to 30 carbon atoms; with the proviso that when R¹ is isononyl, R² is not geranyl.

In particular implementations, resorcinol and its derivatives and/or analogs include compounds having Formula (XIII), wherein R1 is (a) a straight or branched alkyl of 7 to 12 carbon atoms; (b) a group —O—R3, where R3 is a straight or branched alkyl of 5 to 9 carbon atoms, or a straight or branched alkyl substituted at the terminal carbon atom by a phenyl group; or (c) a group —(CH2)n-O-alkyl, where n is an integer from 1 to 7 and the alkyl group contains 1 to 5 carbon atoms.

In particular implementations, resorcinol and its derivatives and/or analogs include compounds of Formula (XIII), wherein R2 is a non-cyclic terpenoid carbon chain such as geranyl, farnesyl, and related non-cyclic terpenes and their isomers as well as other non-cyclic paraffinic or olefinic carbon chains.

In particular implementations, resorcinol and its derivatives and/or analogs include compounds of Formula (XIII), wherein R1 is dimethylheptyl and R2 is geranyl.

In particular implementations, dexanabinol compounds and their derivatives and/or analogs include high enantiomeric purity compounds having Formula (XIV),

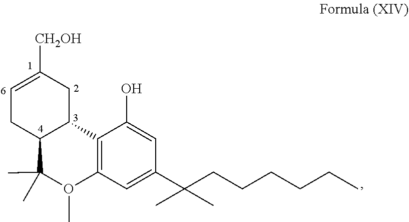

and having the (3S, 4S) configuration and being in enantiomeric excess of at least 99.90% over the (3R,4R) enantiomer.

In particular implementations, cannabimimetic lipid amide compounds and their derivatives and/or analogs include compounds having Formula (XV),

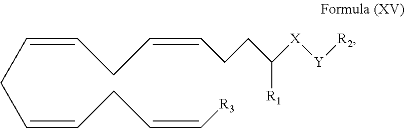

wherein:
X is one of the group consisting of C═O and NH, and Y is the other of that group. Expressed another way, X may be C═O and Y may be NH, or Y may be C═O and X may be NH, but both X and Y may not be the same group.
R₁ is H or an alkyl group. In particular implementations, R₁ is H, CH₃, or (CH₃)₂;
R₂ is an alkyl, a substituted alkyl, an alkenyl or an alkynyl group. In particular implementations, R₂ is CH(R) CH₂Z, CH₂CH(R)Z, or CH(R)(CH₂)nCH₂Z; R being H, CH, CH₃, CHCH, CH₂CF₃, or (CH₃)₂; Z being H, halogen, N₃, NCS, or OH; and n being selected from the group consisting of 0, 1 and 2.
R₃ is an alkyl, a substituted alkyl, an aryl, an alkylaryl, an O-alkyl, an O-alkylaryl, a cyclic and a heterocyclic group. O-alkyl and O-alkylaryl refer to groups in which an oxygen atom is interposed between carbon atoms on the anandamide portion and substituent group.

Examples of such R₃ groups include cyclohexyl, cyclopentyl, alkylcyclohexyl, alkylcyclopentyl, piperidinyl, morpholinyi and pyridinyl. In particular implementations, R₃ is n-C₅H₁₀Z′, n-C₆H₁₂Z′, n-C₇H₁₄Z′, or 1′, 1′-C(CH₃)₂(CH₂)₅ CH₂Z′; Z′ being H, halogens, CN, N₃, NCS, or OH.

In particular implementations, cannabimimetic lipid amide compounds and their derivatives and/or analogs include compounds having Formula (XVI),

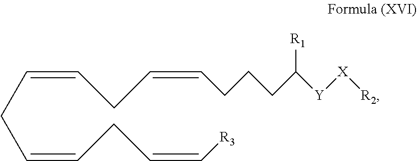

wherein:
Y is one of the group consisting of C═O and NH and X is the other of that group.
R₁ is H or an alkyl group. In particular implementations, R₁ is H, CH₃, or (CH₃)₂.
R₂ is an alkyl, a substituted alkyl, an alkenyl, an alkynyl, an O-alkyl, a cyclic, a polycyclic, or a heterocyclic group. In particular implementations, R₂ is

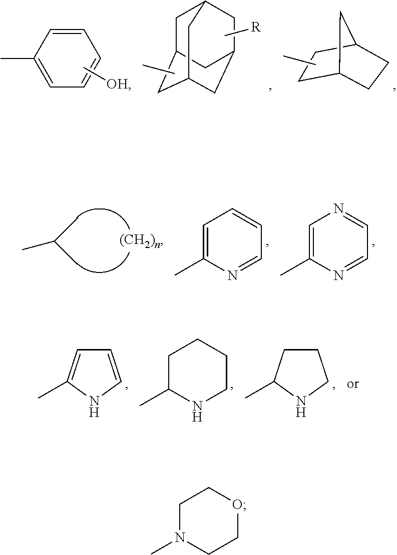

CH═CH₂, CH═C(CH₃)₂, C═CH, CH₂OCH₃, CH(R)(CH₂)nCH₂Z, or CH₂CH(R)(CH₂)nZ;
R being H, CH₃ or (CH₃)₂; Z being H, halogens, N₃, NCS, OH, or OAc; and n 0, 1, or 2; and
R₃ is an alkyl, a substituted alkyl, an aryl, an alkylaryl, an O-alkyl, an O-alkylaryl, a cyclic, or a heterocyclic group. In particular implementations R₃ includes cyclohexyl, cyclopentyl, alkylcyclohexyl, alkylcyclopentyl, piperidinyl, morpholinyi and pyridinyl. In particular implementations, R₃ is n-C₅H₁₀Z′, n-CH₁₂Z′, n-C₇H₁₄Z′, or 1′,1′-C(CH₃)₂CH₂)₅ CH₂Z′; Z′ being H, halogen, CN, N₃, NCS, or OH.

In particular implementations, nabilone and its derivatives and/or analogs include compounds having Formula (XVII):

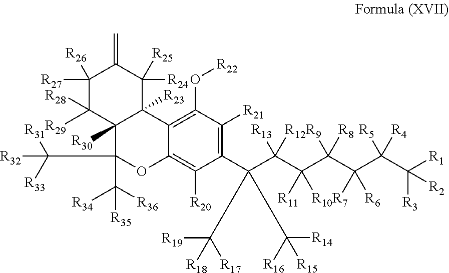

wherein:
R¹-R³⁶ are independently selected from the group consisting of hydrogen and deuterium. Nabilone derivatives and/or analogs can refer to compounds wherein at least one of R¹-R³⁶ includes deuterium. For the chemical structure of nabilone, see FIG. 3.

Synthetic cannabinoids can be medicinal compounds and/or can be provided in combination with supplemental materials (also referred to as “nutritional supplements”). Synthetic cannabinoids are provided in therapeutically-effective amounts to treat a condition, such as those described within this disclosure. Synthetic cannabinoids in combination with supplemental materials claim a benefit related to a classical nutrient deficiency disease; describes how the supplement is intended to affect the structure or function of the human body; characterizes a documented mechanism by which the supplement acts to maintain such structure or function; and/or describes general well-being associated with consumption of the product. In particular implementations, a supplemental material may not claim to diagnose, mitigate, treat, cure, or prevent a specific disease or class of diseases.

In particular implementations, a concentration of cannabinoid(s) in a substantially homogenous mixture of a cannabinoid and an aqueous solution, or in an infused porous substrate, can range from 0.01 mg/mL to 100 mg/mL or from 5 mg/mL to 50 mg/mL. In particular implementations, the concentration can include 0.01 mg/mL, 0.1 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, or more.

Various cannabinoids described herein may be nonpolar and may therefore be insoluble in an aqueous solution. However, a substantially homogenous mixture of a cannabinoid and an aqueous solution can be produced by pressing the aqueous solution through a porous substrate that is infused with the cannabinoid. In various implementations, the porous substrate can be further infused with a flavorant, and/or one or more additional ingredients.

### Flavorants

Various compositions described herein can include a flavorant. For example, an example mixture of an aqueous solution can include a flavorant as a nonpolar material. The flavorant may include one or more aroma- and/or flavor-conferring agents. For example, when the aqueous solution is a beverage, the flavorant can impact and/or improve the taste and/or smell of the beverage. In various implementations, the flavorant can be generally recognized as safe (GRAS), as defined by the U.S. Food and Drug Administration.

In various examples, suitable flavorants can include an alcohol. For example, a flavorant may include at least one of acetoin, ethanol, 1-propanol, 2-propanol, propylene glycol, glycerol, n-butyl alcohol, iso-butyl alcohol, hexyl alcohol, L-menthol, octyl alcohol, cinnamyl alcohol, heptyl alcohol, 1-pentanol, 3-methyl-1-butanol, anisic alcohol, citronellol, n-decanol, geraniol, 3-hexenol, linalool, nerolidol, 2,6-nonadiene-1-ol, nonyl alcohol, rhodinol, terpineol, borneol, clineol, cuminyl alcohol, 10-undecene-1-ol, or 1-hexadecanol.

In various examples, suitable flavorants can include an aldehyde. For example, a flavorant may include at least one of acetaldehyde, anisic aldehyde, benzaldehyde, methyl-1-propanol, citral, citronellal, n-decanal, ethylvanillin, furfural, heliotropin, heptanal, hexanal, 2-hexenal, 3-phenyl-1-propanal, dodecanal, nonyl aldehyde, octyl aldehyde, phenylacetaldehyde, propanal, vanillin, cinnamic aldehyde, perillaldehyde, or cuminaldehyde.

In various examples, suitable flavorants can include a phenol. For example, a flavorant may include at least one of thymol, methyleugenol, acetyleugenol, safrol, eugenol, isoeugenol, anethole, phenol, methylchavicol, carvacrol, α-bisabolol, fornesol, anisole, or propenylguaethol.

In various examples, suitable flavorants can include an acetate. For example, a flavorant may include at least one of iso-amyl acetate, benzyl acetate, benzylphenyl acetate, n-butyl acetate, cinnamyl acetate, citronellyl acetate, ethyl acetate, eugenol acetate, geranyl acetate, hexyl acetate, hydrocinnamyl acetate, linalyl acetate, octyl acetate, phenylethyl acetate, terpinyl acetate, triacetin, potassium acetate, sodium acetate, or calcium acetate.

In various examples, suitable flavorants can include an acid. For example, a flavorant may include at least one of acetic acid, aconitic acid, adipic acid, formic acid, malic acid, capronic acid, 3-phenyl-1-propionic acid, nonanoic acid, lactic acid, phenoxyacetic acid, phenylacetic acid, valeric acid, iso-valeric acid, cinnamic acid, citric acid, mandelic acid, tartaric acid, fumaric acid, tannic acid, or any of their physiologically acceptable salts.

In various examples, suitable flavorants can include a terpene. For example, a flavorant may include at least one of camphor, limonene, or β-caryophyllene.

In various examples, suitable flavorants can include an acetal. For example, a flavorant may include at least one of acetaldehyde dibutyl acetal, acetaldehyde dipropyl acetal, acetaldehyde phenethyl propyl acetal, cinnamic aldehyde ethylene glycol acetal, decanal dimethyl acetal, heptanal dimethyl acetal, heptanal glyceryl acetal, and benzaldehyde propylene glycol acetal.

Example flavorants include, natural and synthetic flavor oils, flavoring aromatics, extracts from plants, leaves, flowers, nuts, fruits, and combinations thereof. Such flavorants include anise oil, cinnamon oil, vanilla, vanillin, cocoa, chocolate, natural chocolate flavor, menthol, grape, lavender, rose, almond, maple syrup, cinnamon, chestnut, hazelnut, coconut, macadamia nut, pecan, peppermint oil, oil of wintergreen, clove oil, bay oil, anise oil, eucalyptus, thyme oil, cedar leaf oil, oil of nutmeg, oil of sage, oil of bitter almonds, cassia oil; citrus oils, such as lemon, orange, lime and grapefruit oils; and fruit essences, including apple, pear, peach, berry, wildberry, date, banana, blueberry, blackberry, guava, grapefruit, kiwi, strawberry, raspberry, currant, cherry, orange (e.g., blood orange), plum, pineapple, pumpkin, kiwi, longan, lychee, mango, passion fruit, ube, taro, fig, melon, and apricot. In particular implementations, flavorants that may be used include natural berry extracts and natural mixed berry flavor, as well as citric and malic acid. In some cases, compositions include flavorants that are extracts and/or oils of nuts, such as of almond, bitter almond, chestnut, hazelnut, macadamia nut, pecan, or a combination thereof.

In particular implementations, concentration of flavorant(s) in a substantially homogenous mixture of a cannabinoid and an aqueous solution can range from 0.1 ug/mL to 100 mg/mL or from 5 mg/mL to 50 mg/mL. In particular implementations, the concentration can include 0.1 ug/mL, 1.0 ug/mL, 10 ug/mL, 100 ug/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, or more.

Various flavorants described herein may be nonpolar and may therefore be insoluble in an aqueous solution. However, a substantially homogenous mixture of a flavorant and an aqueous solution can be produced by pressing the aqueous solution through a porous substrate that is infused with the flavorant. In various implementations, the porous substrate can be further infused with a cannabinoid, and/or additional ingredients.

### Dispersal Agents

Various implementations of the present disclosure relate to compositions that include one or more dispersal agents. In various cases, a porous substrate can be infused with a dispersal agent, a mixture of an aqueous solution and a nonpolar material can include a dispersal agent, a fluid used to infuse a porous substrate can include a dispersal agent, or the like. The term “dispersal agent,” as used herein, can refer to any material that increases the kinetic stability of an emulsion when mixed with the emulsion. In various implementations, a dispersal agent can be food-safe. In various examples, a dispersal agent can include an amphipathic material. The amphipathic material can include a molecule and/or a particle that includes a polar (e.g., hydrophilic) portion and a nonpolar (e.g., hydrophobic) portion.

According to various cases, a dispersal agent can include a surfactant. In various cases, a polar fluid and a nonpolar fluid may resist mixing due to their polarities, such that an interface may be spontaneously generated between the polar fluid and the nonpolar fluid. A surfactant may refer to an amphipathic material that is spontaneously disposed at the interface between a polar fluid and a nonpolar fluid. In some cases, a surfactant can lower surface tension at the interface. In various implementations, a surfactant includes at least one of sodium lauryl sulfate, hydroxylated soy lecithin, polysorbates, or block copolymers of propylene oxide and ethylene oxide. Surfactants can be ionic and/or non-ionic.

Example surfactants can include hydrophilic non-ionic surfactants, such as one or more of alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof. For instance, example hydrophilic surfactants include, but are not limited to, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, poloxamers, and any of their derivatives and/or analogues.

Various implementations of the present disclosure can include lipophilic surfactants. Various examples of lipophilic surfactants include, for instance, polyglucosides, alkyl poly(ethylene oxide), alkyl polyglucosides, fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; mixtures thereof; or their derivatives and/or analogues.

In some implementations, surfactants can be zwitterionic and/or cationic. Examples of such surfactants include, but are not limited to the bile acids (e.g., cholic acid, chenodeoxycholic acid, glycocholic acid, glycodeoxycholic acid, taurocholic acid, taurochenodeoxycholic acid, taurolithocholic acid, deoxycholic acid, lithocholic acid, and ursodeoxycholic acid and salts thereof, e.g., sodium, potassium, lithium), natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methyl cellulose), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether), poly(vinyl-pyrrolidone), diethylene glycol monolaurate, triethanolamine oleate, sodium oleate, potassium oleate, ethyl oleate, oleic acid, ethyllaurate, sodium lauryl sulfate, cetrimonium bromide, cetylpyridinium chloride, benzalkonium chloride, docusate sodium, combinations thereof; analogues thereof, and derivatives thereof.

For example, the lipophilic surfactant can be one or more glycerol fatty acid esters, propylene glycol fatty acid esters, or a mixture thereof, or one or more hydrophobic transesterification products of a polyol with at least one member of the group consisting of vegetable oils, hydrogenated vegetable oils, and triglycerides. Among the lipophilic transesterification products, transesterification products of a polyol (e.g., ethylene glycol, glycerol, propylene glycol, or sorbitol) can be used. As is known in the art, a large number of surfactants of different degrees of hydrophobicity or hydrophilicity can be prepared by reaction of alcohols or polyalcohols with a variety of natural and/or hydrogenated oils. The oils used can include castor oil or hydrogenated castor oil, or an edible vegetable oil such as corn oil, olive oil, peanut oil, palm kernel oil, apricot kernel oil, or almond oil. Alcohols include glycerol, propylene glycol, ethylene glycol, polyethylene glycol, maltol, sorbitol, and pentaerythritol. Among these alcohol-oil transesterified surfactants, hydrophobic surfactants include PEG-5 hydrogenated castor oil, PEG-7 hydrogenated castor oil, PEG-9 hydrogenated castor oil, PEG-6 corn oil (Labrafil® M 2125 CS available from Gattefossé Corporation of Paramus, N.J.), PEG-6 almond oil (Labrafil® M 1966 CS), PEG-6 apricot kernel oil (Labrafil® M 1944 CS), PEG-6 olive oil (Labrafil® M 1980 CS), PEG-6 peanut oil (Labrafil® M 1969 CS), PEG-6 hydrogenated palm kernel oil (Labrafil® M 2130 BS), PEG-6 palm kernel oil (Labrafil® M 2130 CS), PEG-6 triolein (Labrafil® M 2735 CS), PEG-8 corn oil (Labrafil® M WL 2609 BS), PEG-20 corn glycerides (Crovol M40), and PEG-20 almond glycerides (Crovol A40).

In various implementations, a dispersal agent can be infused into a porous substrate, and/or mixed into an aqueous solution, in the form of a substantially spherical structure, such as a liposome and/or micelle. For example, a membrane of the spherical structure can include the dispersal agent. In some cases, the membrane can include a single layer or a bilayer of the dispersal agent. In various implementations, the membrane can encapsulate a nonpolar molecule and/or material (e.g., an oil including a cannabinoid), such that the outer surface of the spherical structure is substantially hydrophilic (e.g., due to the presence of the polar portions of the dispersal agent) and the inner surface of the membrane is substantially nonpolar (e.g., due to the presence of the nonpolar portions of the dispersal agent). Spherical structures can be generated by high-shear mixing of the dispersal agent. In some cases, a nonpolar fluid (e.g., including a nonpolar material, such as a cannabinoid) can be sonicated in the presence of the dispersal agent, thereby generating micelles in which the nonpolar fluid is encapsulated in a membrane of the dispersal agent, wherein the outer surfaces of the micelles are substantially hydrophilic. Accordingly, in some implementations, the spherical structures can improve the emulsification of the nonpolar fluid in an aqueous solution (e.g., coffee, tea, or the like). In various implementations, multiple spherical structures in mixtures and/or porous substrates can have a size of 100 micrometers, 10 micrometers, 1 micrometer, 100 nanometers, 10 nanometers, 1 nanometer, or some other distance. For instance, the spherical structures can have sizes of between 1 nanometer and 100 nanometers. In some cases, the spherical structures can have a size of between 10 nanometers to 100 micrometers. The sizes of the structures can be defined as the hydrodynamic diameter or the hydrodynamic radius of the structures, which may be calculated by applying the Stokes-Einstein equation to photon correlation spectroscopy measurements (e.g., by calculating a zeta average).

According to some implementations, the dispersal agent can be infused into a porous substrate and/or mixed into an aqueous solution, in the form of a liquid crystal, such as a lipid-based lyotropic liquid crystal (see, e.g., Rajabalaya, et al., Drug Des. Devel. Ther. 2017; 11:393-40). A lipid-based lyotropic liquid crystal has an amphiphilic mesogen, in various examples. An example liquid crystal may include a combination of a nonpolar material (e.g., a cannabinoid and/or a flavorant), an aqueous phase, and an anisotropic surfactant (e.g., glyceryl monooleate, monoolein, and/or phytantriol). In some cases, the example liquid crystal further includes a lipid (e.g., an oil). In various examples, the liquid crystal includes a composition of glyceryl monooleate, a poloxamer (e.g., poloxamer 407), and water; and/or a composition of phytantriol, a poloxamer, and water.

### Additional Ingredients

In particular implementations, compositions include additional ingredients. In some examples, these additional ingredients are included (e.g., infused) within a porous substrate and/or a fluid for infusing the porous substrate. For instance, the additional ingredients are transferred from an infused porous substrate to an aqueous solution when the aqueous solution flows through (or otherwise contacts) the infused porous substrate. Some example compositions include a nonpolar additional ingredient.

In various examples, additional ingredients include one or more supplemental materials (also referred to as “supplemental ingredients” or “nutritional supplements”). A supplemental material may include a dietary supplement, a nutritional supplement, a nutraceutical, a food additive, or the like. According to some implementations, a supplemental material is a flavorant. In some cases, a supplemental material includes one or more herbs, spices, and/or other plant-based materials. For instance, example supplemental materials include at least one of acai (e.g., fruit of Euterpe oleracea), agave (e.g., sap, leaves and/or flowers of genus Agave), blueberry (e.g., fruit of Vaccinium corymbosum), chamomile (e.g., Matricaria recutita and/or Chamaemelum nobile), cinnamon (e.g., bark of the genus Cinnamomum), cocoa (e.g., seeds of Theobroma cacao), cranberry (e.g., fruit of Vaccinium oxycoccos and/or Vaccinium macrocarpon), echinacea (e.g., Echinacea purpurea), garlic (e.g., Allium sativum), Gaultheria procumbens, ginger (e.g., root of Zingiber officinale), Ginkgo biloba, ginseng (e.g., Panax ginseng, Panax notoginseng, Panax quinquefolius, etc.), Glycyrrhiza root, goji berry (e.g., fruit of Lycium barbarum and/or Lycium chinense), grape (e.g., seeds and/or fruit of Vitis vinifera), guarana (e.g., seeds of Paullinia cupana), Huperzia serrata, kola nut (e.g., seeds of Cola acuminata and/or Cola nitida), lemon (e.g., fruit of Citrus limon), maca (e.g., root of Lepidium meyenii), Melissa officinalis, mint (e.g., genus Mentha, such as Mentha spicata), Silybum marianum, monkfruit (e.g., fruit of Siraitia grosvenorii), rosehip (e.g., fruit of genus Rosa), rosemary (e.g., leaves of Salvia Rosmarinus), saw palmetto (e.g., Serenoa repens), stevia (e.g., leaves of Stevia rebaudiana), pomegranate (e.g., fruit and/or seeds of Punica granatum), skullcap (genus Scutellaria), soy (e.g., seeds of Glycine max), St. John's wort (e.g., Hypericum perforatum), tea (e.g., green tea leaves), turmeric (e.g., root of Curcuma longa), Valeriana officinalis, yerba mate (e.g., stem and/or leaves of Ilex paraguariensis), or any extract thereof. In some cases, the supplemental ingredients include insect-based materials, such as bee pollen, honey, propolis, or any combination thereof.

In various implementations, a supplemental material includes one or more curcuminoids, such as at least on of curcumin, demethoxycurcumin, or bidemethoxycurcumin. The curcuminoid(s), in some examples, are naturally derived. For example, one or more curcuminoids may be extracted from the root of Curcuma longa.

In various implementations, a supplemental material includes one or more vitamins. Examples of vitamins include vitamin A (e.g., retinol, retinal, alpha carotene, beta-carotene, gamma carotene, cryptoxanthin, or any combination thereof), vitamin B (e.g., thiamine, riboflavin, niacin, pantothenic acid, pyridoxine, pyridoxine 5′-phosphate, pyridoxal, pyridoxal 5′-phosphate, pyridoxamine, pyridoxamine 5′-phosphate, 4-pyridoxic acid, pyritinol, biotin, folic acid, cobalamins, or any combination thereof), vitamin C (e.g., L-ascorbic acid), vitamin D (e.g., cholecalciferol, ergocalciferol, 22-dihydroergocalciferol, sitocalciferol, or any combination thereof), vitamin E (e.g., alpha-tocopherol, beta-tocopherol, gamma-tocopherol, delta-tocopherol, tocopheryl acetate, or any combination thereof), vitamin K (e.g., phylloquinone, menaquinone, or a combination thereof), or any combination thereof. In various implementations, a supplemental material includes one or more minerals. For example, a supplemental material can include calcium (e.g., calcium carbonate, calcium citrate, calcium gluconate, or the like), chromium (e.g., chromium(III) picolinate), copper (e.g., copper gluconate), iodine (e.g., potassium iodate, calcium iodate, or the like), iron (e.g., iron(II) sulfate), magnesium (e.g., magnesium oxide, magnesium citrate, magnesium chloride, or the like), manganese (e.g., manganese gluconate), selenium (e.g., selenomethionine, sodium selenite, sodium selenite, or a combination thereof), zinc (e.g., zinc gluconate, zinc sulfate, zinc acetate, or the like), or a combination thereof. In some cases, a supplemental material includes one or more amino acids hormones, and/or enzymes. For instance, compositions include glucosamine, chondroitin sulfate, L-theanine, melatonin, or any combination thereof.

In some examples, compositions include additional ingredients that can impact an administration effect of the active ingredient and/or compositions. In some examples, compositions include one or more entourage-restoring molecules. Entourage-restoring molecules can refer to molecules that, when provided in a composition with THC, restore or enhance particular desired effects, as compared to the effects of THC alone. In particular implementations the one or more entourage-restoring molecules can include additional cannabinoids, terpenes, flavonoids, and/or aroma and flavor conferring volatiles.

In particular implementations the entourage-restoring molecules include additional cannabinoids (in addition to the primary cannabinoids THC and/or CBD). Cannabis produces over 60 different cannabinoids (Brenneisen, Marijuana and the Cannabinoids, Ch. 2, 2007, Humana Press). Cannabinoids are made by the trichome secretory glands of cannabis, which are highly concentrated in the flowers of female plants. Cannabinoids can also be found in other parts of the cannabis plant, including the stems and leaves.

Cannabinoids other than THC and CBD contribute to the various physiological effects of cannabis. For example, cannabigerol (CBG) counteracts THC-induced paranoia and is anti-inflammatory, anti-bacterial, and anxiolytic. Cannabichromene (CBC) is anti-inflammatory and analgesic. Cannabinol (CBN), which is produced by the degradation of THC, is analgesic, anxiolytic, and has mildly psychoactive effects. Tetrahydrocannabivarin (THCV), in contrast to THC, is an appetite suppressant.

Cannabinoid content can vary widely depending on plant strain, age, growth conditions, and storage conditions. Cannabis strains vary in their non-THC, non-CBD cannabinoid content. For example, whereas the strain Purple Kush contains 0.02% CBN; 0.4% CBG; 0.1% THCV; 0.05% CBC; and 0.1% CBL, the strain Durban Poison contains 0.1% CBN; 1% CBG; 1% THCV; 0.05% CBC; and 1.2% CBL (averages reported by Steep Hill Labs, Inc.). The variation in cannabinoid content across cannabis strains contributes to the distinct entourage effects of each strain.

In particular implementations, the entourage-restoring molecules include one or more of Δ8-tetrahydrocannabinol (Δ8-THC), Δ11-tetrahydrocannabinol (Δ11-THC), CBG, CBC, CBN, cannabinodiol (CBDL), cannabicyclol (CBL), cannabivarin (CBV), THCV, cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), and tetrahydrocannabivarinic acid (THCVA). Examples of these cannabinoids are shown in FIG. 3.

In particular implementations, the entourage-restoring molecules include terpenes. Terpenes can refer to terpenes or terpenoids, or derivatives and/or analogs thereof. Terpenes are a large class of organic molecules that include one or more units of isoprene (C₅H₈). Terpene molecules that include additional functional groups are also known as terpenoids. The isoprene units of terpenes can be linked together to form linear molecules or rings. Terpenes can be classified by the number of isoprene subunits present. For example, hemiterpenes contain one isoprene subunit, monoterpenes contain two isoprene subunits, sesquiterpenes contain three isoprene subunits, and diterpenes contain four isoprene subunits.

In particular implementations, the entourage-restoring molecules include one or more cannabis-derived terpenes. More than 100 terpenes have been identified in cannabis plants (Rothschild et al., Bot J Linn Soc. 2005. 147(4):387-397 and Brenneisen “Forensic Science and Medicine: Marijuana and the Cannabinoids” Chapter 2, ed. M EISohly, Humana Press New York, N.Y., 2007). Like cannabinoids, terpenes are produced by cannabis trichome glands, which are concentrated in cannabis flowers. However, terpenes can also be found in other part of the cannabis plant, such as in stems and leaves. Examples of cannabis-derived terpenes include β-myrcene, α-pinene, β-pinene, linalool, d-limonene, β-caryophyllene, caryophyllene oxide, nerolidol, phytol, ocimene, terpinolene, terpinene, humulene, carene, bisabolol, valencene, elemene, farnesene, menthol, geraniol, guaiol, camphene, camphor, eucalyptol, pulegone, and phellandrene. In particular implementations, the one or more terpenes include alloaromadendrene, (Z)-α-cis-bergamotene, (Z)-α-trans-bergamotene, β-bisabolol, epi-α-bisabolol, β-bisabolene, borneol (camphol), cis-γ-bisabolene, borneol acetate (bornyl acetate), α-cadinene, cis-carveol, α-humulene (α-caryophyllene), γ-cadinene, Δ-3-carene, caryophyllene oxide, 1,8-cineole, citral A, citral B, α-copaene (aglaiene), γ-curcumene, β-cymene, β-elemene, γ-elemene, eucalyptol, α-eudesmol, β-eudesmol, γ-eudesmol, eugenol, cis-β-farnesene (O-β-farnesene), trans-α-farnesene, trans-β-farnesene, trans-γ bisabolene, fenchone, fenchol (norbornanol, β-fenchol), α-guaiene, ipsdienol, lemenol, d-limonene, linalyl alcohol (β-linolool), α-longipinene, menthol, γ-muurolene, trans-nerolidol, nerol, β-ocimene (cis-ocimene), α-phellandrene, 2-pinene, sabinene, cis-sabinene hydrate (cis-thujanol), β-selinene, α-selinene, γ-terpinene, isoterpine, terpineol (α-terpineol), terpineol-4-ol, α-terpinene (terpinene), α-thujene (origanene), viridiflorene (ledene), and/or α-ylange.

In particular implementations, the terpenes include linalool. Linalool is a monoterpene that naturally occurs in many plants including cannabis, lavender, bay laurel, citrus fruits, and mint, among others. Linalool naturally exists as two isomers, known as licareol and coriandrol. Multiple studies have demonstrated anti-inflammatory (Peana et al., Phytomedicine 2002. 9(8):721-6), analgesic (Peana et al., Eur J Pharmacol 2003 460(1):37-41) and anti-anxiety (Linck et al., Phytomedicine 2002. 17(8-9):679-83; Souto-Major et al., Pharmacol Biochem Behav 2011. 100(2):259-63) effects of linalool. Linalool is commonly used as a food additive and is Generally Recognized as Safe by the FDA.

In particular implementations, the terpenes include nerolidol. Nerolidol is a cannabis-derived terpene that has sedative properties (Binet et al., Ann Pharm Fr 1972. 30:611-616). Therefore, nerolidol contributes to the sedative effects of particular cannabis strains.

In particular implementations, the terpenes include pinene. Pinene is a monoterpene that exists as two isomers, α-pinene and β-pinene. Pinene has a pine-like scent and naturally occurs in pine trees and cannabis. Pinene has anti-inflammatory effects (Gil et al., Pharmazie 1989. 44(4):284-7), anti-microbial properties (Nissen et al., Fitoterapia 2010. 81(5):413-19), and is a bronchodilator at low concentration (Falk et al., Scand J Work Environ Health 1990. 16:372-378). Pinene may also improve memory (Perry, et al, Journal of Pharmacy and Pharmacology 2000. 52(7):895-902), and therefore is thought to counteract the short-term memory impairment that can be induced by THC.

In particular implementations, the terpenes include caryophyllene (or β-caryophyllene). β-caryophyllene is a sesquiterpene that naturally occurs in rosemary, hops, cannabis, cloves, black pepper, lavender, caraway, basil, and cinnamon. B-caryophyllene has anti-inflammatory (Gertsch et al., PNAS. 2008. 105(26):9099104), anti-analgesic (Katsuyama et al., European Journal of Pain. 2013. 17(5): 664-675), neuroprotective (Guimaraes-Santos, J Evid Based Complementary Altern Med. 2012. 1-9), anti-anxiety and anti-depressant (Bahi et al., Physiology & Behavior. 2014. 135:119-124) effects. Caryophyllene is a direct agonist of the cannabinoid receptor CB2, which is present on immune cells, and can enhance the anti-inflammatory properties of cannabis.

In particular implementations, the terpenes include limonene. Limonene is a monoterpene that naturally occurs in citrus trees, citronella grass, verbena plants, and cannabis. Limonene is the main component of citrus fruit that confers the citrus-like aroma. Limonene has anti-inflammatory (Piccinelli et al. Life Sci. 2016 S0024-3205(16):30669-5) and anti-depressant effects (Komori et al., 1995). Limonene is commonly used as a food additive and is Generally Recognized as Safe by the FDA.

In particular implementations, the terpenes include β-myrcene. β-myrcene is a monoterpene that is commonly found in hops, parsley, thyme, bay leaves, mangoes, lemongrass and cannabis. β-myrcene has analgesic (Paula-Freire et al., Planta Med. 2016; 82(3):211-6), anti-inflammatory (Lorenzetti et al., J of Ethnopharmacology. 1991.34(1):43-48), anti-microbial (Yoshihiro et al., Natural Medicines. 2004. 58(1), 10-14), and sedative (Rao et al., J Pharm Pharmacol. 1990. 42(12): 877-878) effects. Indica strains of cannabis are characteristically high in β-myrcene (>0.5%), and β-myrcene contributes to the sedating, “couch-lock” inducing effects of indica or indica-dominant strains.

In particular implementations the entourage-restoring molecules include flavonoids. Flavonoids are a class of secondary metabolite found in plants and fungus that each contain a 15-carbon skeleton including two phenyl rings and a heterocyclic ring. Flavonoids can be classified into three groups: i) bioflavonoids or flavonoids, ii) isoflavonoids, and iii) neoflavonoids.

Flavonoids that naturally occur in cannabis plants include cannaflavin A, cannaflavin B, cannaflavin C, vitexin, isovitexin, apigenin, kaempferol, quercetin, luteolin, cinnamaldehyde, and orientin. See FIG. 4 for exemplary structures of particular flavonoids.

In particular implementations, the entourage-restoring molecules include cannaflavin A, cannaflavin B, and/or cannaflavin C. Cannaflavins are flavonoids that are unique to cannabis plants. Cannaflavin A and cannaflavin B both have anti-inflammatory activity (Barrett et al., Experientia 1986. 15; 42(4):452-3).

In particular implementations, the entourage-restoring molecules include apigenin. Apigenin is a flavonoid that naturally occurs in many plants, such as cannabis, parsley, celery and chamomile. Apigenin is an opioid receptor agonist and has many beneficial health effects, including specifically inducing death of cancer cells, anxiolytic activity (Salgueiro et al., Pharmacol Biochem Behav 1997. 58, 887-891) and stimulation of neurogenesis.

In particular implementations, the entourage-restoring molecules include kaempferol. Kaempferol is a flavonoid commonly found in many plant-based foods, including apples, grapes, tomatoes, potatoes, onions, broccoli, squash, cucumber, and berries. There are a wide range of positive health effects of ingesting kaempferol. For example, kaempferol has antioxidant, anti-inflammatory, antimicrobial, anti-cancer, cardioprotective, neuroprotective, antidiabetic, anti-osteoporotic, anxiolytic, analgesic, and antiallergic properties (Calderon-Montano et al., Mini Rev Med Chem. 2011. 11(4):298-344).

In particular implementations, the entourage-restoring molecules include quercetin. Quercetin is a flavonoid found in many plants, including cannabis, kidney beans, capers, cilantro, onion, kale, plum, cranberry, and sweet potato. Quercetin may have antioxidant and anti-cancer effects (Alam et al., Environ Sci Pollut Res Int 2016).

In particular implementations, the entourage-restoring molecules include orientin. Orientin is a flavonoid that can be found in cannabis, passion flower, Acai palm, barley, and millet. Medicinal properties of orientin include antioxidant, antiaging, antimicrobial, anti-inflammatory, vasodilatation, radiation protective, neuroprotective, antidepressant, anti-adipogenesis, and antinociceptive effects (Lam et al., Adv Pharmacol Sci. 2016. 2016:4104595).

In particular implementations the entourage-restoring molecules include other cannabis-derived molecules. In addition to terpenes and flavonoids, certain volatile compounds confer distinct aroma and flavor profiles to cannabis. Examples of aroma and flavor conferring volatiles that are present in cannabis are listed in Rice & Koziel. PLoS One. 2015. 10(12):e0144160. Particular cannabis-derived molecules that contribute to cannabis aroma and flavor include 2-heptanone, methyl heptanoate, methyl salicylate, methyl anthranilate, and hexanal. These molecules are volatile compounds, meaning that they have a high tendency to vaporize. Therefore, aroma and flavor conferring volatile compounds of cannabis are often lost during production of cannabis extracts for human consumption. The molecules 2-heptanone, methyl heptanoate, methyl salicylate, methyl anthranilate, and hexanal, as well as other volatile cannabis-derived compounds, are FDA-approved food additives. In particular implementations, low concentrations (1% or lower) of these volatiles can be used to confer particular aromas and flavors to oral compositions described herein.

In particular implementations the compositions include one or more primary cannabinoids (e.g., THC) and one or more entourage-restoring molecules. Extraction and decarboxylation of THC for oral consumption can lead to loss of entourage effect molecules. Therefore, compositions with THC can be supplemented with cannabis-derived molecules to restore entourage effects.

In particular implementations, entourage-restoring molecules can be obtained from commercially available sources. Many terpenes and flavonoids, such as linalool, β-myrcene, α-pinene, β-pinene, caryophyllene, quercetin, and apigenin are commercially available from various sources. Examples of companies that provide food grade terpenes and flavonoids include Sigma Aldrich, True Terpenes, and NHR Organic Oils. Examples of companies that provide aroma and flavor conferring volatiles include Sigma Aldrich, Eastman Chemical Company, Foodchem International Corporation, and Aurochemicals.

In particular implementations, the supplemental materials and/or entourage-restoring molecules are synthetically produced.

In particular implementations, the curcuminoids are synthetically produced. Examples for synthetic production of curcuminoids can be found in Wichitnithad et al., Molecules. 2011. 16(2). 1888-1900.

In particular implementations, the vitamins are synthetically produced. Examples for synthetic production of vitamin A and vitamin E can be found in Mercier et al., Pure & Appl. Chem. 66(7). 1994. 1509-1518.

In particular implementations, the cannabinoids can be synthetically produced. Examples of techniques for synthetic production of cannabinoids can be found in US2016/0355853; JP2016/509842; Petrzilka et al., Helv Chim Acta. 1967. 50(2):719-723; Kobayashi et al., Org Lett. 2006. 8(13):2699-2702; and Mechoulam & Gaoni, J Am Chem Soc. 1965. 87(14):3273-3275.

In particular implementations, terpenes can be synthetically produced. Examples of techniques for synthetic production of terpenes can be found in US2004/0161819; and WO2006134523. In particular implementations, organisms can be genetically altered to overexpress particular terpenes and the terpenes can be isolated from the organism. Examples of techniques for obtaining terpenes from a genetically modified organism can be found in WO20061111924 and US2010/0297722.

In particular implementations, the flavonoids can be synthetically produced. Exemplary techniques to synthesize flavonoids can be found in Mamoalosi & Van Heerden, Molecules. 2013. 18: 4739-4765 and Wagner & Farkas, The Flavonoids. Chapter: Synthesis of Flavonoids. 1975. 127-213. Springer.

In particular implementations, the supplemental materials and/or entourage-restoring molecules are derived from vegetable matter. Vegetable matter is matter produced by a plant and includes any whole plant or plant part (e.g., bark, wood, leaves, stems, roots, flowers, fruits, seeds, or parts thereof) and/or exudates or extracts thereof. In particular implementations, the compositions can include botanical products. Botanical products can include plant materials, algae, macroscopic fungi, and/or combinations thereof. In particular implementations, the compositions include a mixture of various types of vegetable matter.

In particular implementations, the supplemental materials and/or entourage-restoring molecules can be prepared by pulverization, decoction, expression, and extraction of a starting plant product. The term “extract” can include all of the many types of preparations containing some or all of the active ingredients found in the relevant plants. Extracts may be produced by cold extraction techniques using a variety of different extraction solvents including water, fatty solvents (such as olive oil), and alcoholic solvents (e.g. 70% ethanol). Cold extraction techniques are typically applied to softer parts of the plant such as leaves and flowers, or in cases wherein the desired components of the plant are heat-labile (e.g., terpene) or have a low boiling point (e.g., volatiles). Alternatively, the aforementioned solvents may be used to produce extracts of the desired plants by a hot extraction technique, wherein said solvents are heated to a high temperature, the precise value of said temperature being dependent on the properties of the chosen solvent, and maintained at that temperature throughout the extraction process. Hot extraction techniques are more commonly applied to the harder, tougher parts of the plant, such as bark, woody branches and larger roots. In some cases, sequential extractions can be performed in more than one solvent, and at different temperatures. The plant extract may be used in a concentrated form. Alternatively, the extract may be diluted as appropriate to its intended use.

Additional procedures for producing plant extracts (including hot extraction, cold extraction and other techniques) are described in publications including “Medicinal plants: a field guide to the medicinal plants of the Land of Israel (in Hebrew), author: N. Krispil, Har Gilo, Israel, 1986” and “Making plant medicine, author: R. Cech, pub. by Horizon Herbs, 2000”.

In particular implementations, the additional cannabinoids (non-THC, non-CBD cannabinoids), can be provided in a THC-containing cannabis extract. Cannabis extracts (e.g. CO2 or BHO extracts) can be rich in cannabinoids and preserve the cannabinoid content of the cannabis strain used for extraction. Cannabinoid-rich cannabis extracts are commercially available from a variety of sources.

In particular implementations, supplemental materials, terpenes, flavonoids, and/or aroma and flavor conferring volatiles can be extracted from plants. Exemplary techniques for extracting terpenes from plants can be found in Breitmaier, Terpenes: Flavors, Fragrance, Pharmaca, Pheromones. Ch. 10. 2006. John Wiley & Sons, WO2013174854 and CN101439074. An exemplary technique for obtaining a flavonoid-rich plant extract can be found in Victorio et al., Ecl. Quinn. 2009. 34(1):29-24. Techniques for extracting aroma and flavor conferring volatile compounds include cold-pressing and ethanol extraction.

In particular implementations, the supplemental materials are obtained from plants. For example, one or more curcuminoids are obtained from extracts of Curcuma longa. In some cases, vitamin A is extracted from mango (e.g., fruit of Mangifera indica), papaya (e.g., fruit of Carica papaya), carrots (e.g., root of Daucus carota), one or more squashes (e.g., fruit of genus Cucurbita), sweet potatoes (e.g., root of Ipomoea batatas), yellow corn (e.g., Zea mays), or a combination thereof. In some examples, compositions include palm oil, which is a source of vitamin A. In various examples, vitamin E is extracted from avocado (e.g., Persea americana), asparagus (e.g., genus Asparagus), beet greens (e.g., leaves of Beta vulgaris), collard greens (e.g., leaves of Brassica oleracea), mango, peanut (e.g., Arachis hypogaea), red bell pepper (e.g., fruit of Capsicum annuum), spinach (e.g., leaves of Spinacia oleracea), sunflower seeds (e.g., seeds of Helianthus annuus), or a combination thereof. In some examples, compositions include peanut oil, safflower oil, and/or soybean oil, which are sources of vitamin E.

In particular implementations, the entourage-restoring molecules are obtained from extracts of plants other than cannabis. In particular implementations, entourage-restoring molecules are obtained from any plant that produces the desired molecule. For example, vitexin is a cannabis-derived flavonoid that is also found in the plants hawthorn and passionflower and therefore vitexin can be obtained from hawthorn or passionflower extract.

In particular implementations, the total concentration of supplemental materials (e.g., vitamin A, vitamin E, turmeric, one or more curcuminoids, etc.) in a composition can be at 0.01 ug/mL or ug/mg, 0.1 ug/mL or ug/mg, 1 ug/mL or ug/mg, 10 ug/mL or ug/mg, 50 ug/mL or ug/mg, 100 ug/mL or ug/mg, 200 ug/mL or ug/mg, 300 ug/mL or ug/mg, 400 ug/mL or ug/mg, 500 ug/mL or ug/mg, 600 ug/mL or ug/mg, 700 ug/mL or ug/mg, 800 ug/mL or ug/mg, 900 ug/mL or ug/mg, 1 mg/mL or mg/mg, 10 mg/mL or mg/mg, 50 mg/mL or mg/mg, 100 mg/mL or mg/mg, 200 mg/mL or mg/mg, 300 mg/mL or mg/mg, 400 mg/mL or mg/mg, 500 mg/mL or mg/mg, 600 mg/mL or mg/mg, 700 mg/mL or mg/mg, 800 mg/mL or mg/mg, 900 mg/mL or mg/mg, 1 g/mL or g/mg, or 10 g/mL or g/mg. In some cases, a composition includes an amount of vitamin A that is no more than 110 mg and is greater than or equal to 500 ug, such as an amount of vitamin A in a range of 700 ug to 32 mg. In some examples, a composition includes an amount of vitamin E that is no more than 130 mg and is greater than or equal to 10 mg, such as an amount of vitamin E in a range of 15 mg to 40 mg. In some examples, an amount of one or more curcuminoids in a composition is no more than 20 g and is greater than or equal to 50 ug, such as an amount of curcuminoid(s) in a range of 200 mg to 6 g.

In particular implementations, compositions are created by combining one or more primary cannabinoids (such as THC) with one or more supplemental materials and/or one or more entourage-restoring molecules.

In particular implementations, the relative amount of each cannabis-derived molecule in the formulation can be chosen to mimic the entourage effects of a particular cannabis strain. Cannabis strains can be tested to quantify primary cannabinoids and entourage molecules of a strain. The quantities of various cannabis-derived molecules present in a cannabis sample can be determined by analytical laboratory techniques, such as mass spectrometry, gas chromatography, or high-performance liquid chromatography. Chemical profiling of cannabis strains is routinely performed by commercial testing laboratories, such as Steep Hill Labs, Inc., The Werc Shop, SC Labs and Analytical 360. Examples of terpenes quantified by commercial cannabis profiling labs include limonene, β-myrcene, caryophyllene, α-pinene, β-pinene, bisabolol, humulene, linalool, and terpinolene. Cannabinoids analyzed by commercial cannabis testing laboratories include THC, CBD, CBV, THCA, THCV, CBN, CBDA, CBL and CBG.

In particular implementations, the primary cannabinoids and the entourage-restoring molecules are combined at a ratio that mimics their ratio in a particular cannabis strain, as measured through analytical testing. For example, compositions can be created to mimic the entourage effects of the strain Sour Diesel, for which a cannabinoid and terpene analysis is publicly availably (as a Strain Fingerprint®, Steep Hill Labs, Inc., Oakland Calif.). Sour Diesel can contain an average of 20% THC, 0.2% CBD, 0.5% CBG, 0.3% CBL, 0.3% β-myrcene, 0.3% limonene, and 0.25% caryophyllene. Therefore, a composition with restored Sour Diesel entourage effects can be created by combining 100 mg THC, 1 mg CBD, 2.5 mg CBG, 1.5 mg CBL, 1.5 mg β-myrcene, 1.5 mg limonene, and 1.25 mg caryophyllene, which mimics the strain's relative concentration of each of these components, providing a restored entourage effect compared to an otherwise equivalent composition lacking one or more of the entourage-restoring molecules. Other cannabis strains with publicly available cannabinoid and terpene analyses include Super Lemon Haze, Agent Orange, Berry While, Blue Dream, Cherry Pie, Durban Poison, Grape Ape, and Purple Kush.

In particular implementations, the total concentration of primary cannabinoid(s) (e.g., THC) in a composition can be at 1 ug/mL or ug/mg, 10 ug/mL or ug/mg, 50 ug/mL or ug/mg, 100 ug/mL or ug/mg, 200 ug/mL or ug/mg, 300 ug/mL or ug/mg, 400 ug/mL or ug/mg, 500 ug/mL or ug/mg, 600 ug/mL or ug/mg, 700 ug/mL or ug/mg, 800 ug/mL or ug/mg, 900 ug/mL or ug/mg, 950 ug/mL or ug/mg, 1 mg/mL or mg/mg, 5 mg/mL or mg/mg, 10 mg/mL or mg/mg, 20 mg/mL or mg/mg, 30 mg/mL or mg/mg, 40 mg/mL or mg/mg, 50 mg/mL or mg/mg, 60 mg/mL or mg/mg, 70 mg/mL or mg/mg, 80 mg/mL or mg/mg, 90 mg/mL or mg/mg, 100 mg/mL or mg/mg, 200 mg/mL or mg/mg, 300 mg/mL or mg/mg, 400 mg/mL or mg/mg, 500 mg/mL or mg/mg, 600 mg/mL or mg/mg, 700 mg/mL or mg/mg, 800 mg/mL or mg/mg, 900 mg/mL or mg/mg, or 950 mg/mL or mg/mg.

In particular implementations, the ratio (w/w) of the primary cannabinoids to each entourage-restoring molecule in a composition can be 1000:1, 500:1, 200:1, 100:1, 50:1, 20:0, 10:1, 1:1, 0.2:1, or 0.1:1. For example, a composition with 5 mg CBD and 5 mg THC (10 mg total primary cannabinoids) with 1 mg β-myrcene would have a 10:1 primary cannabinoid: β-myrcene ratio. In particular implementations, the ratio of the primary cannabinoids to any entourage-restoring molecule can be chosen based on their ratio in any cannabis strain.

### Experimental Example

In this example, brewed products of espresso were prepared using porous substrates infused with CBD oils. In each experiment, round porous substrates including cellulose fiber paper (Whatman filter paper grade 2) were prepared. Each of the porous substrates had a diameter of 2.25 inches. Each infused porous substrate was prepared by dispensing a 165 uL volume sample of CBD oil on a porous substrate. Each CBD oil included sesame oil (Banyan Botanicals) and pure CBD dissolved in the sesame oil. In each experiment, an infused porous substrate was disposed in a VST portafilter basket for a Lamarcocco GS3 espresso machine. The basket was then loaded with 15 g (±1 g) of fine ground espresso beans that were tamped flat in the portafilter basket at a pressure of 20 to 30 pounds (Ibs) and the loaded portafilter basket was attached to the espresso machine. The espresso machine had a boiler temperature of 199° F. In each experiment, the espresso machine pressed heated water through the loaded portafilter basket, thereby emitting the brewed espresso product.

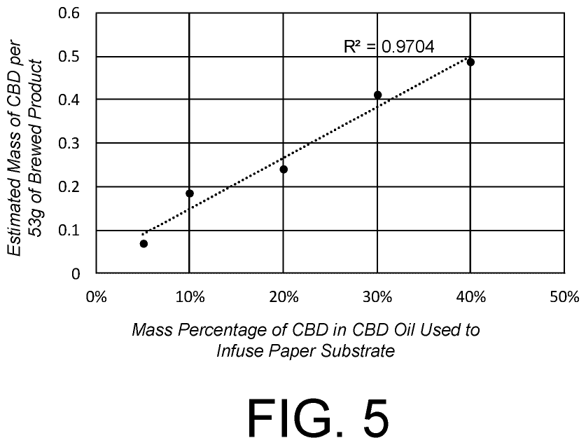

A first set of experiments was performed using a first set of porous substrates infused with CBD oils having different strengths. The CBD oils used for the first set of experiments were prepared with strengths including 5 (mass) % CBD, 10 (mass) % CBD, 20 (mass) % CBD, 30 (mass) % CBD, and 40 (mass) % CBD. A brewed product was collected for each experiment using a brew pressure of 11 bar. A sample of each brewed product was tested for CBD content. An estimated mass of the CBD in each brewed product was calculated by multiplying the mass percentage of CBD in the sample by the mass of the total brewed product. Table 1 and FIG. 5 shows the result of the first set of experiments.

TABLE 1
Results of the first set of experiments

| | | | | | | | | Estimated Mass |
|---|---|---|---|---|---|---|---|---|
| | Mass of | | | Mass CBD | Mass % | Estimated | Estimated | of CBD in Brewed |
| Mass % of | Brewed | Sample | Mass CBD | per Mass | CBD in | Mass of CBD in | Mass of CBD | Product Compared |
| CBD in | Product | Mass | in Sample | of Sample | Sample | Brewed Product | per 53 g of | to 5% CBD Oil |
| CBD Oil | (g) | (g) | (mg) | (mg/g) | (g/g) | (mg) | Brewed Product | Experiment |
| 5% | 53.0 | 10.186 | 0.693 | 0.068 | 0.0068 | 3.6040 | 0.0680 | 1.0000 |
| 10% | 55.6 | 10.171 | 1.800 | 0.177 | 0.0177 | 9.8412 | 0.1857 | 2.7306 |
| 20% | 56.6 | 10.186 | 2.302 | 0.226 | 0.0226 | 12.7916 | 0.2414 | 3.5493 |
| 30% | 51.8 | 10.177 | 4.285 | 0.421 | 0.0421 | 21.8078 | 0.4115 | 6.0510 |
| 40% | 74.0 | 10.123 | 3.533 | 0.349 | 0.0349 | 25.8260 | 0.4873 | 7.1659 |

The results of the first set of experiments illustrate that the mass of CBD in the brewed product is positively correlated with the amount of CBD dispensed on the porous substrate used to create the brewed product. As shown in FIG. 5, there is a substantially linear relationship between the mass percentage of CBD in the CBD oil and the estimated mass of CBD per 53 g of brewed product.

In addition, a second set of experiments was performed, wherein a first set of porous substrates was infused with CBD oils having different strengths. The CBD oils were prepared with strengths including 5 (mass) % CBD, 10 (mass) % CBD, 20 (mass) % CBD, 30 (mass) % CBD, and 40 (mass) % CBD. A brewed product was collected for each experiment using a brew pressure of 11 bar. A sample of each brewed product was tested for CBD content. The mass percentage of CBD in each sample of the second set of experiments was compared to the mass percentage of CBD in the corresponding experiment in the first set of experiments. For example, the mass percentage of CBD in the sample prepared using the 5% CBD oil in the second set of experiments was compared to the mass percentage of CBD in the sample prepared using the 5% CBD oil in the first set of experiments. Table 2 shows the result of the second set of experiments:

TABLE 2
Results of the second set of experiments

| | | | Comparing |
|---|---|---|---|
| | Mass | | Mass CBD |
| | CBD per | Mass % | of Sample |
| Mass % | Mass of | CBD in | to First |
| of CBD in | Sample | Sample | Set of |
| CBD Oil | (mg/g) | (g/g) | Experiments |
| 5% | 0.066 | 0.0065 | 97.1% |
| 20% | 0.237 | 0.0233 | 98.2% |
| 30% | 0.409 | 0.4010 | 99.4% |
| 40% | 0.520 | 0.0511 | 106.7% |

The results of the second set of experiments illustrate the relative reproducibility of the results of the first set of experiments.

A third set of experiments was performed, wherein a third set of porous substrates was infused with a CBD oil having a strength of 10 (mass) % CBD. A first brewed product was collected using a brew pressure of 7 bar and a second brewed product was collected using a brew pressure of 11 bar. A sample of each brewed product was tested for CBD content. An estimated mass of the CBD in each brewed product was calculated by multiplying the mass percentage of CBD in the sample by the mass of the total brewed product. Table 3 shows the result of the third set of experiments:

TABLE 3
Results of the third set of experiments

| | Mass of | | | Mass CBD | Mass % | Estimated | Estimated |
|---|---|---|---|---|---|---|---|
| Brew | Brewed | Sample | Mass CBD | per Mass | CBD in | Mass of CBD in | Mass of CBD |
| Pressure | Product | Mass | in Sample | of Sample | Sample | Brewed Product | per 53 g of |
| (bar) | (g) | (g) | (mg) | (mg/g) | (g/g) | (mg) | Brewed Product |
| 7 | 36.6 | 10.274 | 3.072 | 0.299 | 0.0299 | 10.9434 | 0.2065 |
| 11 | 58.7 | 10.0 | 1.860 | 0.186 | 0.0186 | 10.9182 | 0.2060 |

The results of the third set of experiments show that brew pressures of 7 bar and 11 bar can result in different masses of brewed product. However, the 7 bar and 11 bar brew pressures resulted in relatively insignificant changes in the mass of CBD transferred to the respective brewed products.

### Example Clauses

The following example clauses provide various implementations of the present disclosure:
1. A method including generating a substantially homogenous mixture of an aqueous solution and a nonpolar material by flowing the aqueous solution through a porous substrate infused with the nonpolar material.
2. The method of example 1, wherein the aqueous solution is pressed at a pressure in a range of 7 bars to 11 bars.
3. The method of example 1 or 2, wherein the aqueous solution is pressed through the porous substrate by a pressure generated by a pump.
4. The method of any one of examples 1 to 3, wherein the aqueous solution is pressed through the porous substrate by a hydrostatic pressure.
5. The method of any one of examples 1 to 4, wherein the aqueous solution includes at least one of coffee, espresso, or tea.
6. The method of any one of examples 1 to 5, further including generating the aqueous solution by exposing water to plant matter including at least one of coffee grounds, espresso grounds, tea leaves, herbal leaves, or a spice.
7. The method of example 6, wherein the plant matter is disposed in at least one of a portafilter, a drip coffee basket, a French press, or a beverage pod.
8. The method of any one of examples 1 to 7, wherein the aqueous solution has a temperature in a range of 160 degrees Fahrenheit (° F.) and 210° F.
9. The method of any one of examples 1 to 8, further including adding, to the substantially homogenous mixture, at least one of nitrogen gas, a carbonated liquid, steam, a dairy product, a dairy substitute, a sugar, a syrup, or an alcohol.
10. The method of any one of examples 1 to 9, wherein the nonpolar material includes a cannabinoid.
11. The method of example 10, wherein the cannabinoid includes at least one of delta-8-tetrahydrocannabinol (THC), delta-9-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).
12. The method of any one of examples 1 to 11, wherein the porous substrate includes at least one of a flavorant, a mineral, a vitamin, vitamin A, vitamin E, turmeric, or a curcuminoid.
13. The method of example 12, wherein the flavorant includes at least one of anise oil, cinnamon oil, vanilla, vanillin, cocoa, chocolate, natural chocolate flavor, menthol, grape, peppermint oil, oil of wintergreen, clove oil, bay oil, anise oil, eucalyptus, hazelnut oil, thyme oil, cedar leaf oil, oil of nutmeg, oil of sage, oil of bitter almonds, cassia oil, lemon oil, orange oil, lime oil, or grapefruit oil.
14. The method of any one of examples 1 to 13, wherein the substantially homogenous mixture includes a dispersal agent.
15. The method of example 14, wherein the dispersal agent includes a surfactant.
16. The method of example 14 or 15, wherein the substantially homogenous mixture includes a liposome and/or a micelle including the nonpolar material and the dispersal agent, the dispersal agent encapsulating the nonpolar material.
17. The method of example 16, wherein a diameter of the liposome and/or the micelle is in a range of 10 nanometers (nm) to 100 micrometers (um).
18. The method of example 16 or 17, wherein the substantially homogenous mixture includes a liquid crystal, the liquid crystal including the liposome and/or the micelle.
19. The method of example 18, wherein the liquid crystal includes a lipid-based lyotropic liquid crystal.
20. A method including:
placing, on a porous substrate, a predetermined volume of a fluid including a nonpolar material;
placing, in basket, the porous substrate including the nonpolar material; and
generating a substantially homogenous mixture of an aqueous solution and the at least one cannabinoid by flowing the aqueous solution through the basket.
21. The method of example 20, wherein the porous substrate includes at least one of a cannabinoid, vitamin A, vitamin E, turmeric, or a curcuminoid.
22. The method of example 21, wherein the predetermined volume includes 1 milligram to 7.0 grams of the cannabinoid.
23. The method example 21 or 22 wherein the cannabinoid includes at least one of tetrahydrocannabinol (THC), delta-9-THC, delta-8-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).
24. The method of any one of examples 21 to 23, wherein the cannabinoid includes CBD.
25. The method of any one of examples 20 to 24, wherein the predetermined volume includes a solvent including at least one of ethanol, an aqueous solution of ethanol, an animal butter, coconut oil, grape seed oil, olive oil, palm oil, papaya seed oil, peanut oil, hazelnut oil, sesame oil, sprouted wheat oil, or wheat germ oil.
26. The method of any one of examples 20 to 25, wherein the predetermined volume includes 0.1 milliliters (mL) to 5.0 mL.
27. The method of any one of examples 20 to 26, wherein, in response to placing the predetermined volume, the fluid is distributed through the porous substrate via capillary action.
28. The method of any one of examples 20 to 27 further including: in response to placing the fluid, drying the fluid.
29. The method of any one of examples 20 to 28, further including: placing, in the basket, coffee grounds.
30. The method of any one of examples 20 to 29, wherein the porous substrate includes at least one of paper, plant fiber, ceramic, polymer, or metal.
31. A system including:
a basket disposed at least partially around an interior space;
plant matter disposed in the interior space; and
an infused porous substrate disposed in the interior space, the infused porous substrate including a nonpolar material.
32. The system of example 31, wherein the plant matter includes at least one of coffee grounds, espresso grounds, tea leaves, herbal leaves, or a spice.
33. The system of example 31 or 32, wherein the basket includes a beverage pod, the beverage pod including:
an outer cup-shaped container; and
a filter basket disposed in a space at least partially enclosed by the outer container, and wherein the plant matter and the infused porous substrate are disposed inside of the filter basket.
34. The system of any one of examples 31 to 33, further including:
a reservoir disposed at a higher altitude than the basket, wherein a hydrostatic pressure of fluid in the reservoir causes the fluid to flow into the interior space.
35. The system of any one of examples 31 to 34, further including a pump configured to generate a pressure that pushes a fluid through the interior space.
36. The system of example 35, wherein the pump is configured to generate the pressure in a range of 5 bars to 16 bars.
37. The system of example 35 or 36, wherein the fluid pump includes a rotary pump or a vibration pump.
38. The system of any one of examples 34 to 37, further including a multi-way valve disposed between an outlet of the pump and an inlet of the basket.
39. The system of example 38, wherein the multi-way valve is configured to block the fluid from flowing from the pump to the basket when the pressure is less than a threshold pressure.
40. The system of example 39, wherein the threshold pressure is 5 bars to 16 bars.
41. The system of any one of examples 31 to 40, further including a heater configured to heat the fluid.
42. The system of example 41, wherein the heater includes at least one of thermoblock, a thermocoil, or a boiler.
43. The system of any one of examples 31 to 42, wherein the basket includes a portafilter including stainless steel.
44. The system of any one of examples 31 to 43, wherein the infused porous substrate includes at least one of paper, a plant fiber, ceramic, a polymer, or a metal.
45. The system of any one of examples 31 to 44, wherein the nonpolar material includes at least one of a cannabinoid, a flavorant, a mineral, a vitamin, vitamin A, vitamin E, turmeric, or a curcuminoid.
46. The system of example 45 wherein the cannabinoid includes at least one of tetrahydrocannabinol (THC), delta-9-THC, delta-8-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).
47. The system of example 46 wherein the at least one cannabinoid includes CBD and/or THC.
48. The system of any one of examples 44 to 47, wherein the flavorant includes at least one of hazelnut oil, anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leaf oil, oil of nutmeg, oil of sage, oil of bitter almonds, cassia oil, lemon oil, orange oil, lime oil, or grapefruit oil.
49. A system configured to perform the method of any one of examples 1 to 30.
50. A system for generating an infused beverage, the system including:
a basket at least partially enclosing an interior space;
coffee grounds disposed in the interior space;
an infused paper substrate disposed in the interior space of the basket, the infused paper substrate including cellulose and 5 to 70 milligrams (mg) of cannabidiol (CBD) and/or tetrahydrocannabinol (THC).
51. The system of example 50, further including:
a heater configured to heat water to a temperature that is greater than or equal to 70 degrees Celsius (° C.) and less than or equal to 100° C.;
a pump configured to flow the heated water into the basket at a pressure that is greater than or equal to 7 bars and that is less than or equal to 11 bars.
52. The system of example 50 or 51, wherein the basket includes a stainless steel portafilter.
53. The system of any one of examples 50 to 52, wherein the basket includes a beverage pod, the beverage pod including:
an outer cup-shaped container;
a lid attached to a rim of the outer cup-shaped container; and
a filter basket disposed in a space at least partially enclosed by the outer container, and wherein the coffee grounds and the infused porous substrate are disposed inside of the filter basket.
54. The system of any one of examples 50 to 53, wherein the infused paper substrate has a thickness of 100 to 300 micrometers (um) and a width of 40,000 to 80,000 um.
55. The system of any one of examples 50 to 54, wherein the infused paper substrate further includes at least one of vitamin A, vitamin E, turmeric, or a curcuminoid.
56. An infused porous substrate for generating an infused beverage, the substrate including:
cellulose paper; and
5 to 70 milligrams (mg) of at least one cannabinoid.
57. The infused porous substrate of example 56, wherein the cellulose paper has a thickness of 100 to 300 micrometers (um) and a width of 40,000 to 80.00 um.
58. The infused porous substrate of example 57, further including at least one of vitamin A, vitamin E, turmeric, or a curcuminoid.
59. The infused porous substrate of example 57 or 58, wherein the infused porous substrate is single-use and/or disposable.
60. A method for generating a substantially homogenous mixture of a fluid and a cannabinoid by flowing the fluid through a paper substrate infused with 5 to 70 milligrams (mg) of the cannabinoid.
61. The method of example 60, wherein a volume of the substantially homogenous mixture includes 40 to 70 mL.
62. The method of example 60 or 61, wherein a mass of the cannabinoid in the substantially homogenous mixture includes 2.5 milligrams (mg) to 50 mg.
63. The method of any one of examples 60 to 62, wherein the fluid includes at least one of water, coffee, espresso, tea, cocoa, milk, broth, or cider.
64. The method of any one of examples 60 to 63, wherein the fluid is a polar fluid.
65. The method of any one of examples 60 to 64, wherein the fluid is pressed through the paper substrate at a pressure that is greater than or equal to 7 bars and that is less than or equal to 11 bars.
66. The method of any one of examples 60 to 65, further including: heating the fluid; and
exposing the fluid to a plant material.
67. The method of example 66, wherein the plant material includes at least one of coffee grounds, espresso grounds, tea leaves, herbal leaves, and/or a spice.
68. The method of example any one of examples 60 to 67, wherein the cannabinoid includes at least one of tetrahydrocannabinol (THC), delta-9-THC, delta-8-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).
69. The method of any one of examples 60 to 68, wherein the paper substrate is further infused with at least one of vitamin A, vitamin E, turmeric or a curcuminoid.
70. A beverage pod, including:
an outer cup-shaped container;
a filter basket disposed in a space at least partially enclosed by the outer container; and
an infused porous substrate disposed in an interior space of the filter basket, the infused porous substrate being infused with a nonpolar material.
71. The beverage pod of example 70, wherein the nonpolar material includes at least one of tetrahydrocannabinol (THC), delta-9-THC, delta-8-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).
72. The beverage pod of example 70 or 71, wherein the infused porous substrate further includes at least one of anise oil, cinnamon oil, vanilla, vanillin, cocoa, chocolate, natural chocolate flavor, menthol, grape, peppermint oil, oil of wintergreen, clove oil, bay oil, anise oil, eucalyptus, hazelnut oil, thyme oil, cedar leaf oil, oil of nutmeg, oil of sage, oil of bitter almonds, cassia oil, lemon oil, orange oil, lime oil, grapefruit oil, vitamin D, vitamin E, turmeric, or a curcuminoid.
73. The beverage pod of any one of examples 70 to 72, further including:
a water-soluble powder, a syrup, or plant matter disposed in the interior space of the filter basket, the plant matter including at least one of coffee grounds, tea leaves, herbal leaves, or a spice.

### Terminology and Other Clarifications

As will be understood by one of ordinary skill in the art, each implementation disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. A feature materially affects an implementation, for example, if the feature results in a mixture of an aqueous fluid and a nonpolar material that is not substantially homogenous, as defined herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or implementations of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations of this invention are described herein, including the best mode known to the inventors for carrying out the invention. Of course, variations on these described implementations will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the implementations of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred implementations of the present invention only and are presented in the cause of providing what is believed to be the most useful and readily understood description of the principles and conceptual aspects of various implementations of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art.

What is claimed is:

1. A system for generating an infused beverage, the system comprising:
a basket at least partially enclosing an interior space;
coffee grounds disposed in the interior space;
an infused paper substrate disposed in the interior space of the basket, the infused paper substrate comprising cellulose and about 5 to 70 milligrams (mg) of cannabidiol (CBD).

2. The system of claim 1, further comprising:
a heater configured to heat water to a temperature that is greater than or equal to about 70 degrees Celsius (° C.) and less than or equal to about 100° C.;
a pump configured to flow the heated water into the basket at a pressure that is greater than or equal to about 7 bars and that is less than or equal to about 11 bars.

3. The system of claim 1, wherein the basket comprises a stainless steel portafilter.

4. The system of claim 1, wherein the infused paper substrate has a thickness of 100 to 300 micrometers (um) and a width of about 40,000 to about 80,000 um.

5. The system of claim 1, wherein the infused paper substrate further comprises at least one of vitamin A, vitamin E, turmeric, or a curcuminoid.

6. A beverage pod comprising:
the system of claim 1;
an outer cup-shaped container at least partially disposed around the system; and
a lid attached to a rim of the outer cup-shaped container,
wherein the basket comprises a porous material.

7. An infused porous substrate for generating an infused beverage, the substrate comprising:
cellulose paper; and
about 5 to 70 (mg) of at least one cannabinoid.

8. The infused porous substrate of claim 7, wherein the cellulose paper has a thickness of about 100 to about 300 micrometers (um) and a width of about 40,000 to about 80.00 um.

9. The infused porous substrate of claim 7, further comprising at least one of vitamin A, vitamin E, turmeric, or a curcuminoid.

10. The infused porous substrate of claim 7, wherein the infused porous substrate is single-use and/or disposable.

11. A method for generating a substantially homogenous mixture of a fluid and a cannabinoid by flowing the fluid through a paper substrate infused with about 5 to 70 milligrams (mg) of the cannabinoid.

12. The method of claim 11, wherein a volume of the substantially homogenous mixture comprises about 200 mL to about 550 mL.

13. The method of claim 11, wherein a mass of the cannabinoid in the substantially homogenous mixture comprises about 2.5 milligrams (mg) to about 50 mg.

14. The method of claim 11, wherein the fluid comprises at least one of water, coffee, espresso, tea, cocoa, milk, broth, or cider.

15. The method of claim 11, wherein the fluid is a polar fluid.

16. The method of claim 11, wherein the fluid is pressed through the paper substrate at a pressure that is greater than or equal to about 7 bars and that is less than or equal to about 11 bars.

17. The method of claim 11, further comprising: heating the fluid; and
exposing the fluid to a plant material.

18. The method of claim 17, wherein the plant material comprises at least one of coffee grounds, espresso grounds, tea leaves, herbal leaves, and/or a spice.

19. The method of claim 11, wherein the cannabinoid comprises at least one of tetrahydrocannabinol (THC), delta-9-THC, delta-8-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).

20. The method of claim 11, wherein the paper substrate is further infused with at least one of vitamin A, vitamin E, turmeric, or a curcuminoid.

21. A method comprising generating a substantially homogenous mixture of an aqueous solution and a nonpolar material by flowing the aqueous solution through a porous substrate infused with the nonpolar material.

22. The method of claim 21, wherein the aqueous solution is pressed at a pressure in a range of about 7 bars to about 11 bars.

23. The method of claim 21, wherein the aqueous solution is pressed through the porous substrate by a pressure generated by a pump.

24. The method of claim 21, wherein the aqueous solution is pressed through the porous substrate by a hydrostatic pressure.

25. The method of one of claim 21, wherein the aqueous solution comprises at least one of coffee, espresso, tea, cider, cocoa, fruit juice, vegetable juice, or broth.

26. The method of claim 21, further comprising generating the aqueous solution by exposing water to a plant material comprising coffee grounds, espresso grounds, tea leaves, herbal leaves, and/or a spice.

27. The method of claim 26, wherein the plant material is disposed in at least one of a portafilter, a drip coffee basket, a French press, a pour over basket, or a beverage pod.

28. The method of claim 21, wherein the aqueous solution has a temperature in a range of about 70 degrees Celsius (° C.) to about 100° C.

29. The method of claim 21, further comprising adding, to the substantially homogenous mixture, at least one of nitrogen gas, a carbonated liquid, steam, a dairy product, a dairy substitute, a sugar, a syrup, or an alcohol.

30. The method of claim 21, wherein the nonpolar material comprises a cannabinoid.

31. The method of claim 30, wherein the cannabinoid comprises at least one of tetrahydrocannabinol (THC), delta-9-THC, delta-8-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).

32. The method of claim 21, wherein the nonpolar material comprises a flavorant.

33. The method of claim 32, wherein the flavorant comprises at least one of anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leaf oil, oil of nutmeg, oil of sage, oil of bitter almonds, cassia oil, lemon oil, orange oil, lime oil, or grapefruit oil.

34. The method of claim 21, wherein the nonpolar material comprises at least one of vitamin A, vitamin E, turmeric, or a curcuminoid.

35. The method of claim 21, wherein the substantially homogenous mixture comprises a dispersal agent.

36. The method of claim 35, wherein the dispersal agent comprises a surfactant.

37. The method of claim 36, wherein the substantially homogenous mixture comprises a liposome and/or a micelle comprising the nonpolar material and the dispersal agent, the dispersal agent encapsulating the nonpolar material.

38. The method of claim 37, wherein a diameter of the liposome and/or the micelle is in a range of about 10 nanometers (nm) to about 100 micrometers (um).

39. The method of claim 37, wherein the substantially homogenous mixture comprises a liquid crystal, the liquid crystal comprising the liposome and/or the micelle.

40. A method comprising:
placing, on a porous substrate, a predetermined volume of a fluid comprising a nonpolar material;
placing, in basket, the porous substrate comprising the nonpolar material; and
generating a substantially homogenous mixture of an aqueous solution and the nonpolar material by flowing the aqueous solution through the basket.

41. The method of claim 40, wherein the nonpolar material comprises a cannabinoid.

42. The method of claim 41, wherein the predetermined volume comprises about 0.5 to about 7.0 grams of the cannabinoid.

43. The method claim 41 wherein the cannabinoid comprises at least one of tetrahydrocannabinol (THC), delta-8-THC, delta-9-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).

44. The method of claim 41, wherein the cannabinoid comprises CBD.

45. The method of claim 40, wherein the predetermined volume comprises at least one of ethanol, an aqueous solution of ethanol, an animal butter, coconut oil, grape seed oil, olive oil, palm oil, papaya seed oil, peanut oil, sesame oil, sprouted wheat oil, wheat germ oil, hazelnut oil, vitamin A, vitamin E, turmeric, or a curcuminoid.

46. The method of claim 40, wherein the predetermined volume comprises about 0.1 milliliters (mL) to about 5.0 mL.

47. The method of claim 40, wherein, in response to placing the predetermined volume, the fluid is distributed through the porous substrate via capillary action.

48. The method of claim 40, further comprising: in response to placing the fluid, drying the fluid.

49. The method of claim 40, further comprising: placing, in the basket, coffee grounds.

50. The method of claim 40, wherein the porous substrate comprises at least one of paper, plant fiber, ceramic, polymer, or metal.

51. A system comprising:
a basket disposed at least partially around an interior space;
coffee grounds disposed in the interior space; and
an infused porous substrate disposed in the interior space, the infused porous substrate comprising a nonpolar material.

52. The system of claim 51, further comprising:
a reservoir disposed at a higher altitude than the basket, wherein a hydrostatic pressure of fluid in the reservoir causes the fluid to flow into the interior space.

53. The system of claim 51, further comprising a pump configured to generate a pressure that pushes a fluid through the interior space.

54. The system of claim 53, wherein the pump is configured to generate the pressure in a range of 5 to 12 bars.

55. The system of claim 51, wherein the fluid pump comprises a rotary pump or a vibration pump.

56. The system of claim 51, further comprising a multi-way valve disposed between an outlet of the pump and an inlet of the basket.

57. The system of claim 56, wherein the multi-way valve is configured to block the fluid from flowing from the pump to the basket when the pressure is less than a threshold pressure.

58. The system of claim 57, wherein the threshold pressure is about 5 bars to about 16 bars.

59. The system of claim 51, further comprising a heater configured to heat the fluid.

60. The system of claim 59, wherein the heater comprises at least one of thermoblock, a thermocoil, or a boiler.

61. The system of claim 51, wherein the basket comprises a portafilter comprising stainless steel.

62. The system of claim 51, wherein the infused porous substrate comprises at least one of paper, a plant fiber, ceramic, a polymer, or a metal.

63. The system of claim 51, wherein the nonpolar material comprises a cannabinoid.

64. The system of claim 63 wherein the cannabinoid comprises at least one of tetrahydrocannabinol (THC), delta-9-THC, delta-8-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).

65. The system of claim 63 wherein the cannabinoid comprises CBD.

66. The system of claim 63, wherein the infused porous substrate further comprises at least one of anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leaf oil, oil of nutmeg, oil of sage, oil of bitter almonds, cassia oil, lemon oil, orange oil, lime oil, grapefruit oil, vitamin A, vitamin E, turmeric, or a curcuminoid.

67. A beverage pod comprising:
the system of claim 51;
an outer cup-shaped container at least partially disposed around the system; and
a lid attached to a rim of the outer cup-shaped container,
wherein the basket comprises a porous material.

68. A beverage pod, comprising:
an outer cup-shaped container;
a filter basket disposed in a space at least partially enclosed by the outer container; and
an infused porous substrate disposed in an interior space of the filter basket, the infused porous substrate being infused with a nonpolar material.

69. The beverage pod of claim 68, wherein the nonpolar material comprises at least one of tetrahydrocannabinol (THC), delta-9-THC, delta-8-THC, cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), or cannabidiol (CBD).

70. The beverage pod of claim 68, wherein the infused porous substrate further comprises at least one of anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leaf oil, oil of nutmeg, oil of sage, oil of bitter almonds, cassia oil, hazelnut oil, lemon oil, orange oil, lime oil, grapefruit oil, vitamin D, vitamin E, turmeric, or a curcuminoid.

71. The beverage pod of claim 68, further comprising:
a water-soluble powder, a syrup, or plant matter disposed in the interior space of the filter basket, the plant matter comprising at least one of coffee grounds, tea leaves, herbal leaves, or a spice.
